(19) United States
(12) Patent Application Publication
(10) Pub. No.: US 2023/0233691 A1
(43) Pub. Date: Jul. 27, 2023

(54) METHODS AND COMPOSITIONS FOR TREATING CHRONIC INFLAMMATORY INJURY, METAPLASIA, DYSPLASIA AND CANCERS OF EPITHELIAL TISSUES

(71) Applicants: University of Houston System, Houston, TX (US); Tract Pharmaceuticals, Inc., West Hartford, CT (US)

(72) Inventors: Wa XIAN, Sugar Land, TX (US); Frank MCKEON, Sugar Land, TX (US); Shan WANG, Houston, TX (US); Audrey-Ann LIEW, Houston, TX (US); Matthew VINCENT, Amesbury, MA (US)

(73) Assignees: University of Houston System, Houston, TX (US); Tract Pharmaceuticals, Inc., West Hartford, CT (US)

(21) Appl. No.: 18/048,773

(22) Filed: Oct. 21, 2022

Related U.S. Application Data
(60) Provisional application No. 63/270,762, filed on Oct. 22, 2021.
(60) Provisional application No. 63/315,777, filed on Mar. 2, 2022.

(51) Int. Cl. A61K 47/55; A61K 9/51; A61K 9/06; A61K 9/00; A61K 31/5025; A61P 35/00; A61P 1/00

(52) U.S. Cl. CPC A61K 47/55; A61K 9/51; A61K 9/06; A61K 9/0024; A61K 9/0053; A61K 31/5025; A61P 35/00; A61P 1/00

(57) ABSTRACT

The present disclosure provides methods and formulations for treating a patient suffering from one or more of chronic inflammatory injury, metaplasia, dysplasia or cancer of esophageal tissue and gastric tissue, which method comprises administering to the patient an agent that selectively kills or inhibits the proliferation or differentiation of pathogenic Barrett's Esophagus stem cells (BESCs) or Gastric Intestinal Metaplasia stem cells (GIMSCs) relative to normal regenerative esophageal stem cells or gastric stem cells in the tissue in which the BESCs or GIMSCs are found.

### PRIORITY CLAIM

This application claims benefit of priority to U.S. Provisional Application Ser. No. 63/270,762, filed Oct. 22, 2021 and 63/315,777, filed Mar. 2, 2022, the entire contents of both applications being hereby incorporate by reference.

### STATEMENT REGARDING FEDERALLY FUNDED RESEARCH

This invention was made with government support under Grant Nos. 1R01DK115445-01A1, 1R01CA241600-01 and U24CA228550 awarded by the National Institutes of Health and Grant No. W81XWH-20-1-0755 awarded by the Department of Defense. The government has certain rights in the invention.

### BACKGROUND

Metaplasia is the replacement of one differentiated cell type with another mature differentiated cell type that is not normally present in a specific tissue. Typically, metaplasia is triggered by environmental stimuli, which may act in concert with the deleterious effects of microorganisms and inflammation. A hallmark of metaplasia is a change in cellular identity.

Universally, metaplasia is a precursor to low-grade dysplasia, which can culminate in high-grade dysplasia and carcinoma. See FIG. 7. Typically, the risk of a patient developing cancer increases in a pronounced manner as an inflammatory disease or metaplasia progresses to dysplasia.

The persistence of treatment failures for many cancers is driving interest in preemptive strategies directed at precursor lesions. It is now clear that cancer is a late manifestation of a decades-long evolutionary process which is dominated, at least temporally, by a succession of precursor lesions. For colorectal cancer, this process initiates as small adenomas associated with APC mutations, progresses to large adenomas marked by activating KRAS mutations and a loss of epithelial polarity, and finally, with the acquisition of mutations in genes such as p53 and SMAD4, the onset of invasive cancer. The analogous process for gastric adenocarcinoma linked to chronic H. pylori infections was defined by Correa as a linear path from low- and high-risk gastric intestinal metaplasia (GIM), dysplasia, and invasive cancer, a progression driven in part by the acquisition of mutations in tumor suppressor and proto-oncogenes. Barrett's esophagus (BE), the “intestinal metaplasia” precursor lesion for esophageal adenocarcinoma (EAC), was discovered nearly 70 years ago, and is thought to progress along a path to cancer in a series of steps that parallel the Correa sequence for intestinal gastric adenocarcinoma.

FIG. 8 provides a statistical overview of the risk associated with Barret's Esophagus (BE). BE is the result of chronic gastroesophageal reflux disease (GERD) and represents the end stage of the natural course of this disease. It has been estimated that 20% of the population in the United States suffers from gastroesophageal reflux and that about 10% of these patients are diagnosed with BE. Commonly, BE is discovered during endoscopy for the evaluation of GERD symptoms.

It is documented that longstanding exposure of esophageal mucosa to gastric acidity results in cellular damage of the stratified squamous epithelium and creates an abnormal environment, which stimulates repair in the form of intestinal epithelial metaplasia. The consequence is that the stratified squamous epithelium, which physiologically lines the esophageal mucosa, is replaced by a pathological, specialized columnar epithelium which is neither of cardiac nor of stomach type, but exhibits features of the intestinal type of epithelium. This pathological type of epithelium usually demonstrates DNA alterations that predispose to malignancy. The alterations in BE are histologically classified into three categories, depending on whether or not they exhibit dysplasia: (1) BE without dysplasia; (2) BE with low-grade dysplasia; and (3) BE with high-grade dysplasia (HGD). In BE with HGD, dysplasia is confined to the mucosa without crossing the basement membrane. If dysplasia extends beyond the basement membrane into the lamina propria through the in-coming lymphatic network, it is defined as intramucosal (superficial) adenocarcinoma, whereas if it invades the muscularis mucosa layer it becomes invasive adenocarcinoma. Thus, BE with HGD is considered a precursor of invasive adenocarcinoma.

Six to twenty percent of patients with BE and HGD are at greatest risk of developing adenocarcinoma within a short period of time, ranging from 17 to 35 month at follow-up. Esophagostomy specimens from patients with BE and HGD revealed invasive adenocarcinoma in 30%-40% of cases. A recent meta-analysis demonstrated that patients with BE and HGD developed esophageal adenocarcinoma with an average incidence of 6 every 100 patients per year, during the first 1.5 to 7 years of endoscopic surveillance. Furthermore, the majority of esophageal adenocarcinoma is thought to have evolved from cells that have undergone Barrett's metaplasia.

BE is also classified into two categories according to the extent of intestinal metaplasia above the gastroesophageal junction: (1) long segment BE, if the extent of the intestinal epithelium is greater than 3 cm; and (2) short segment BE, if it is less than 3 cm. Among patients who undergo endoscopy for symptoms of GERD, the incidence of long segment BE is 3%-5%, whereas short segment BE occurs in 10%-15%. Whether long and short segment BE share the same pathogenetic alterations or the same predisposition to malignancy still remains unclear; however, both conditions are currently treated in the same manner.

A common, and invasive, means for treating certain Barrett's Esophagus patients is through endoscopic ablation therapy, such as radiofrequency ablation, photodynamic therapy or cryoablation of esophageal tissue. However, despite a reasonably high percentage of patients that reach remission after therapy, many of those patients relapse within a few years. For other patients, whether because they are refractory to ablative therapy or ineligible due to severe co-morbidities, there are even fewer treatment options and those that exist still leave a significant need for more effective therapies with better results and/or long durations of remission.

Similar metaplasia-to-dysplasia-to-cancer transitions are observed across a variety of other epithelial tissues. Metaplasia tends to occur in tissues constantly exposed to environmental agents, which are often injurious in nature. For example, the pulmonary system (lungs and trachea) and the gastrointestinal tract are common sites of metaplasia owing to their contacts with air and food, respectively. In the ovaries, the dynamic interaction between ovarian surface epithelium and underlying ovarian stroma appears to be the origin of epithelial differentiation, metaplasia and finally malignant transformation.

There is a substantial unmet medical need not only for treatments that are effective for cancers of epithelial tissues, but also treatments directed to metaplasia and dysplasia of those tissues.

### SUMMARY

One aspect of the present invention provides a method for treating a patient suffering from chronic inflammatory injury, metaplasia, dysplasia or cancer of an epithelial tissue, which method comprises administering to the patient an anti-PESC agent that selectively kills or inhibits the proliferation or differentiation of pathogenic epithelial stem cells (PESCs) relative to normal epithelial stem cells in the tissue in which the PESC is found. Representative epithelial tissues include pulmonary, genitourinary, gastrointestinal, pancreatic and hepatic tissues.

For instance, the present disclosure derives by extension from these discrete stem cell population and is premised at least in part on the notion that Barrett's esophagus relies on specific stem cells to all neoplastic lesions involved in the progression to EAC. From patient-matched endoscopic biopsies of Barrett's, dysplasia, and EAC, the inventors demonstrate that each has clonogenic cells that show unlimited proliferative potential and absolute commitment to the neoplastic lesion from which they were derived. Unexpectedly, these stem cell clones proved to be remarkably stable at the level of copy number and single nucleotide variation both in vitro and in vivo. This property enabled an assembly of their phylogenetic relationships to describe, at high resolution, the evolution of EAC, and in turn revealed a discrete intermediate between Barrett's and dysplasia that likely corresponds to the high-risk histological entity termed “low-grade dysplasia”. The present disclosure relates to the exploitation of the adaptability of these clones—both normal regenerative esophageal stem cells and pathogenic esophageal stem cells (an example of a PESCs)-to high-throughput screening platforms to identify drug combinations that selectively kill the PESCs (i.e., the Barrett's pathogenic stem cells) while sparing normal regenerative esophageal stem cells, and show that these same combinations also eliminate patient-matched dysplasia and esophageal cancer stem cells (such as EAC stem cells).

Accordingly, in certain embodiments of the present disclosure provides a method for treating a patient suffering from chronic inflammatory injury, metaplasia, dysplasia or cancer of esophageal tissue, which method comprises administering to the patient an IAP Inhibitor agent that selectively kills or inhibits the proliferation or differentiation of pathogenic esophageal stem cells relative to normal regenerative esophageal stem cells.

In certain embodiments, the IAP Inhibitor is administered in combination with a TAK1 inhibitor.

In certain embodiments, the IAP Inhibitor is administered in combination with a RET inhibitor.

In certain embodiments, the target epithelial tissue is an epithelial-derived tumor, such as an ovarian tumor, a lung tumour, a gastric tumor or an esophageal tumor, or a metastatic site thereof, and the PESC is a cancer stem cell.

Another aspect of the disclosure provides a method of reducing proliferation, survival, migration, or colony formation ability of PESCs in a subject in need thereof comprising contacting the PESC with a therapeutically effective amount of an IAP Inhibitor agent that selectively kills or inhibits the proliferation or differentiation of a PESC population relative to normal regenerative esophageal stem cells in the esophageal tissue in which the PESCs are found.

For example, the present disclosure provides a method for treating a patient suffering from one or more of esophagitis (including Eosinophilic esophagitis or EoE), Barrett's Esophagus, esophageal dysplasia or esophageal cancer, which method comprises administering to the patient an IAP Inhibitor agent that selectively kills or inhibits the proliferation or differentiation of Barrett's Esophagus stem cells (BESC) relative to normal esophageal stem cells. In certain embodiments, the patient presents with esophagitis. In certain embodiments, the patient presents with Barrett's Esophagus. In certain embodiments, the patient presents with esophageal dysplasia. In certain embodiments, the patient presents with esophageal cancer. In certain embodiments, the patient presents with esophageal carcinoma, such as esophageal adenocarcinoma or esophageal squamous cell carcinoma.

Another aspect of the disclosure provides a method of reducing proliferation, survival, migration, or colony formation ability of a BESC in a subject in need thereof comprising contacting the BESC with a therapeutically effective amount of an IAP Inhibitor agent that selectively kills or inhibits the proliferation or differentiation of BESC relative to normal esophageal stem cells.

Another aspect of the invention provides a pharmaceutical preparation for treating one or more of chronic inflammatory injury, metaplasia, dysplasia or cancer of an epithelial tissue, which preparation comprises an anti-PESC agent that selectively kills or inhibits the proliferation or differentiation of PESCs relative to normal epithelial stem cells.

In certain embodiments, the disclosure provides a pharmaceutical preparation for treating one or more of esophagitis, Barrett's esophagus, esophageal dysplasia or esophageal cancer, which preparation comprises an IAP Inhibitor agent that selectively kills or inhibits the proliferation or differentiation of PESCs relative to normal esophageal stem cells. In certain embodiments, the patient presents with esophagitis. In certain embodiments, the patient presents with Barrett's Esophagus. In certain embodiments, the patient presents with esophageal dysplasia. In certain embodiments, the patient presents with esophageal cancer. In certain embodiments, the patient presents with esophageal carcinoma, such as esophageal adenocarcinoma or esophageal squamous cell carcinoma. In certain embodiments, the disclosure provides a pharmaceutical preparation for treating one or more of dysplasia, metaplasia or cancer involving lung tissue, such as for the treatment of non-small cell lung carcinoma (NSCLC) or small cell lung carcinoma (SCLC), which preparation comprises an IAP Inhibitor agent that selectively kills or inhibits the proliferation or differentiation of PESCs involved in the lung disease or disorder.

In certain embodiments, the disclosure provides a pharmaceutical preparation for treating one or more of dysplasia, metaplasia or cancer involving ovarian, fallopian and/or cervical tissue, such as for the treatment of cervical metaplasia, cervical cancer, fallopian cancer and/or ovarian cancer (including taxol and/or cisplatin-resistant ovarian cancer, which preparation comprises an IAP Inhibitor agent that selectively kills or inhibits the proliferation or differentiation of PESCs involved in the ovarian, fallopian and/or cervical disease or disorder

In certain embodiments, the disclosure provides a pharmaceutical preparation for treating one or more of dysplasia, metaplasia or cancer involving gastric tissue, such as for the treatment of gastric metaplasia or gastric cancer, which preparation comprises an IAP Inhibitor agent that selectively kills or inhibits the proliferation or differentiation of PESCs involved in the gastric disease or disorder

Yet another aspect of the disclosure provides a drug eluting device, such as for treating one or more of esophagitis, Barrett's esophagus, esophageal dysplasia or esophageal cancer, which device comprises drug release means including an IAP Inhibitor agent that selectively kills or inhibits the proliferation or differentiation of PESCs relative to normal regenerative esophageal stem cells, which device when deployed in a patient positions the drug release means proximal to the luminal surface of the esophagus and releases the agent in an amount sufficient to achieve a therapeutically effective exposure of the luminal surface to the agent. In certain embodiments, the patient presents with esophagitis. In certain embodiments, the patient presents with Barrett's Esophagus. In certain embodiments, the patient presents with esophageal dysplasia. In certain embodiments, the patient presents with esophageal cancer. In certain embodiments, the patient presents with esophageal carcinoma, such as esophageal adenocarcinoma or esophageal squamous cell carcinoma. Examples of drug eluting devices are drug eluting stents, drug eluting collars and drug eluting balloons.

In other embodiments, there are provided drug eluting devices that can be implanted proximal to the diseased portion of the luminal surface of the esophagus, such as implanted extraluminally (i.e., submucosally or in or on the circular muscle or longitudinal muscle) rather than intraluminally.

In certain embodiments, the IAP Inhibitor agent has an IC₅₀ for selectively killing PESCs that is ⅕^{th} or less the IC₅₀ for killing normal regenerative esophageal stem cells in the tissue in which the PESCs are found, more preferably 1/10^{th}, 1/20^{th}, 1/50^{th}, 1/100^{th}, 1/250^{th}, 1/500^{th} or even 1/1000^{th} or less the IC₅₀ for killing normal regenerative esophageal stem cells.

In certain embodiments, the IAP Inhibitor agent has an IC₅₀ for selectively killing BESCs that is ⅕^{th} or less the IC₅₀ for killing normal esophageal stem cells, more preferably 1/10^{th}, 1/20^{th}, 1/50^{th}, 1/100^{th}, 1/250^{th}, 1/500^{th} or even 1/1000^{th} or less the IC₅₀ for killing normal esophageal stem cells.

In certain embodiments, the IAP Inhibitor agent has an IC₅₀ for selectively inhibiting the proliferation of PESCs that is ⅕^{th} or less the IC₅₀ for inhibiting normal regenerative esophageal stem cells in the tissue in which the PESCs are found, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal regenerative esophageal stem cells.

In certain embodiments, the IAP Inhibitor agent has an IC₅₀ for selectively inhibiting the proliferation of BESCs that is ⅕^{th} or less the IC₅₀ for inhibiting the proliferation of normal esophageal stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal esophageal stem cells.

In certain embodiments, the IAP Inhibitor agent has an IC₅₀ for selectively inhibiting the differentiation of PESCs that is ⅕^{th} or less the IC₅₀ for inhibiting the differentiation of normal regenerative esophageal stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the differentiation of normal regenerative esophageal stem cells.

In certain embodiments, the IAP Inhibitor agent has an IC₅₀ for selectively inhibiting the differentiation of BESCs that is ⅕^{th} or less the IC₅₀ for inhibiting the differentiation of normal esophageal stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the differentiation of normal esophageal stem cells.

In certain embodiments, the IAP Inhibitor agent has a therapeutic index (TI) for treating esophagitis, Barrett's Esophagus, esophageal dysplasia and/or esophageal cancer of at least 2, and more preferably has a therapeutic index of at least 5, 10, 20, 50, 100, 250, 500 or 1000 for treating esophagitis, Barrett's Esophagus, esophageal dysplasia and/or esophageal cancer. In certain embodiments, the combined administration of the anti-PESC agent and the ESO Regenerative agent has a therapeutic index (TI) for treating ovarian, fallopian and or cervical metaplasia or dysplasia of at least 2, and more preferably has a therapeutic index of at least 5, 10, 20, 50, 100, 250, 500 or 1000.

In certain embodiments, the combined administration of the anti-PESC agent and the ESO Regenerative agent has a therapeutic index (TI) for treating ovarian cancer (such as taxol and/or cisplatin resistant ovarian cancer) of at least 2, and more preferably has a therapeutic index of at least 5, 10, 20, 50, 100, 250, 500 or 1000.

In certain embodiments, the combined administration of the anti-PESC agent and the ESO Regenerative agent has a therapeutic index (TI) for treating lung cancer (such NSCLC or SCLC) of at least 2, and more preferably has a therapeutic index of at least 5, 10, 20, 50, 100, 250, 500 or 1000.

In certain embodiments, the combined administration of the anti-PESC agent and the ESO Regenerative agent has a therapeutic index (TI) for treating lung metaplasia or dysplasia of at least 2, and more preferably has a therapeutic index of at least 5, 10, 20, 50, 100, 250, 500 or 1000.

In certain embodiments, the IAP Inhibitor agent inhibits the proliferation or differentiation of PESCs, or kills PESCs, with an IC₅₀ of 10⁻⁶ M or less, more preferably 10⁻⁷ M or less, 10⁻⁸ M or less or 10⁻⁹ M or less.

In certain embodiments, the IAP Inhibitor agent inhibits the proliferation or differentiation of BESCs, or kills BESCs, with an IC₅₀ of 10⁻⁶ M or less, more preferably 10⁻⁷ M or less, 10⁻⁸ M or less or 10⁻⁹ M or less.

In certain embodiments, the IAP Inhibitor agent is administered during or after endoscopic ablation therapy, such as radiofrequency ablation, photodynamic therapy or cryoablation of esophageal tissue.

In certain embodiments, the IAP Inhibitor agent is administered by topical application, such as to esophageal tissue.

In certain embodiments, the IAP Inhibitor agent is administered by submucosal injection, such as into esophageal tissue.

In certain embodiments, the IAP Inhibitor agent is formulated as part of a bioadhesive formulation.

In certain embodiments, the IAP Inhibitor agent is formulated as part of a drug-eluting particle, drug eluting matrix or drug-eluting gel.

In certain embodiments, the IAP Inhibitor agent is formulated as part of a bioerodible drug-eluting particle, bioerodible drug eluting matrix or bioerodible drug-eluting gel.

In certain embodiments, the disclosure provides a esophageal topical retentive formulation for topical application to the luminal surface of the esophagus, comprising (i) an IAP Inhibitor agent that selectively kills or inhibits the proliferation or differentiation of pathogenic epithelial stem cells relative to normal esophageal stem cells, (ii) a bioadhesive, and (iii) optionally, one or more pharmaceutically acceptable excipients.

For instance, the formulation can have a mucosal surface residence half-life on esophageal tissue of at least 30 minutes, more preferably at least 60, 120, 180, 240 or even 300 minutes.

For instance, the formulation can produce at least a minimally effective concentration (MEC) of the IAP Inhibitor agent in the esophageal tissue to which it is applied to which it is applied for at least 30 minutes, more preferably at least 60, 120, 180, 240 or even 300 minutes.

For instance, the formulation can produce IAP Inhibitor agent concentration in the esophageal tissue to which it is applied with T1/2 of at least 2 hours, more preferably at least 4, 6, 8, 10 or even 12 hours.

In certain embodiments, the formulation produces a systemic concentration of the IAP Inhibitor agent which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In certain embodiments, the topical formulation is a viscous bioadhesive liquid to coat the esophagus.

In certain embodiments, the topical formulation comprises anti-PESC eluting multiparticulates, microparticles, nanoparticles or microdiscs

In further embodiments, there is provided bioadhesive nanoparticle having a polymeric surface with an adhesive force equivalent to an adhesive force of between 10 N/m² and 100,000 N/m² measured on human mucosal surfaces, which nanoparticle further includes at least one IAP Inhibitor agent, the IAP Inhibitor agent dispersed therein or thereon, wherein the nanoparticle elutes the IAP Inhibitor agents into the mucous gel layer when adhered to mucosal tissue.

In some embodiments, the IAP Inhibitor is a compound of Formula I:

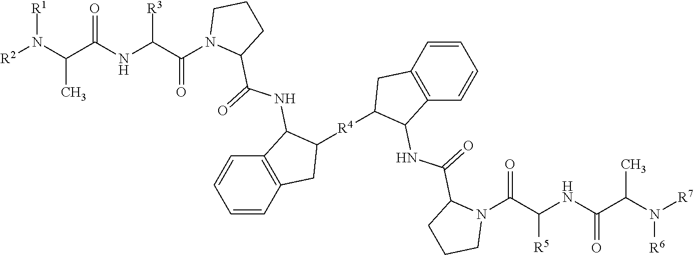

or a pharmaceutically acceptable salt thereof, wherein:

R¹ and R² are independently H or C₍₁₋₆₎alkyl;

R³ is H or C₍₃₋₈₎cycloalkyl;

R⁴ is —OC₍₃₋₁₀₎alkylO—, —OC₍₃₋₁₀₎alkenylO—, or —OC₍₃₋₁₀₎alkynylO—;

R⁵ is H or C₍₃₋₈₎cycloalkyl; and

R⁶ and R⁷ are independently H or C₍₁₋₆₎alkyl.

In some embodiments, one of R¹ and R² is C₍₁₋₆₎alkyl and the other of R¹ and R² is H. In some compounds, one of R¹ and R² is methyl and the other of R¹ and R² is H. In some embodiments, each of R¹ and R² is H.

In some embodiments, R³ is C₍₃₋₈₎cycloalkyl. In some embodiments, R³ is cyclohexyl.

In some embodiments, R⁴ is

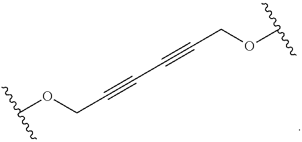 [SMILES: *OCC#CC#CCO*]

In some embodiments, R⁴ is

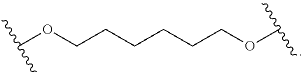 [SMILES: *OCCCCCCO*]

In some embodiments, R⁵ is C₍₃₋₈₎cycloalkyl. In some embodiments, R⁵ is cyclohexyl.

In some embodiments, one of R⁶ and R⁷ is C₍₁₋₆₎alkyl and the other of R⁶ and R⁷ is H. In some embodiments, one of R⁶ and R⁷ is methyl and the other of R⁶ and R⁷ is H. In some embodiments, each of R⁶ and R⁷ is H.

In some embodiments, one of R¹ and R² is C₍₁₋₆₎alkyl, the other of R¹ and R² is H, R³ is C₍₃₋₈₎cycloalkyl, R⁴ is —OC₍₃₋₁₀₎alkynylO—, R⁵ is C₍₃₋₈₎cycloalkyl, one of R⁶ and R⁷ is C₍₁₋₆₎alkyl, and the other of R⁶ and R⁷ is H.

In some embodiments, one of R¹ and R² is methyl and the other of R¹ and R² is H, R³ is cyclohexyl, R⁴ is

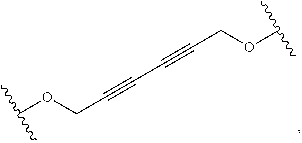 [SMILES: *OCC#CC#CCO*]

R⁵ is cyclohexyl, one of R⁶ and R⁷ is methyl, and the other of R⁶ and R⁷ is H. In some embodiments, the compound of Formula I is selected from

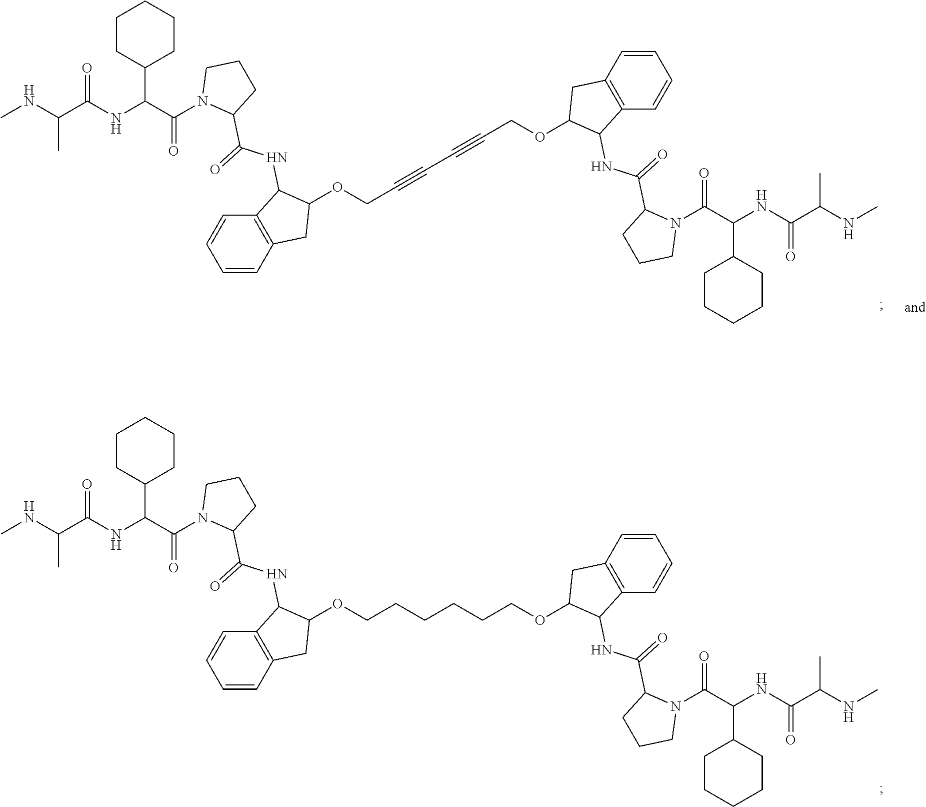

or a pharmaceutically acceptable salt thereof

In some embodiments, the present disclosure provides a compound of Formula Ia:

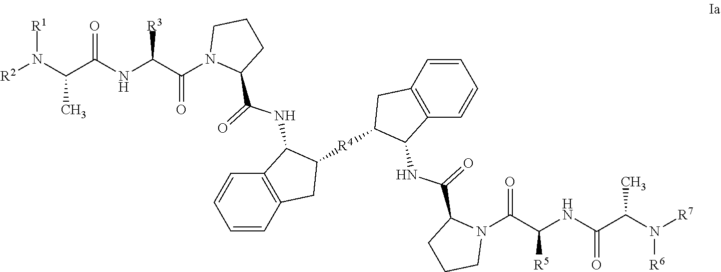

or a pharmaceutically acceptable salt thereof, wherein each of R¹, R², R³, R⁴, R⁵, R⁶, and R⁷ is as defined above and described herein.

In some embodiments, the present disclosure provides a compound of Formula Ib:

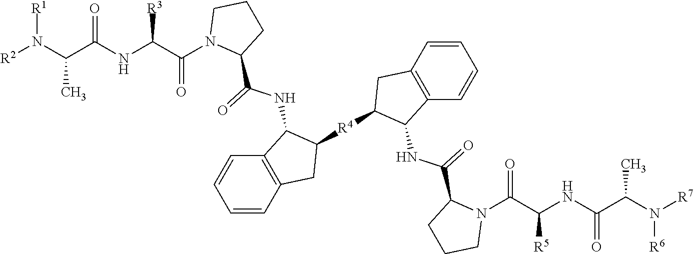

or a pharmaceutically acceptable salt thereof, wherein each of R¹, R², R³, R⁴, R⁵, R⁶, and R⁷ is as defined above and described herein.

In some embodiments, the compound of Formula I is selected from

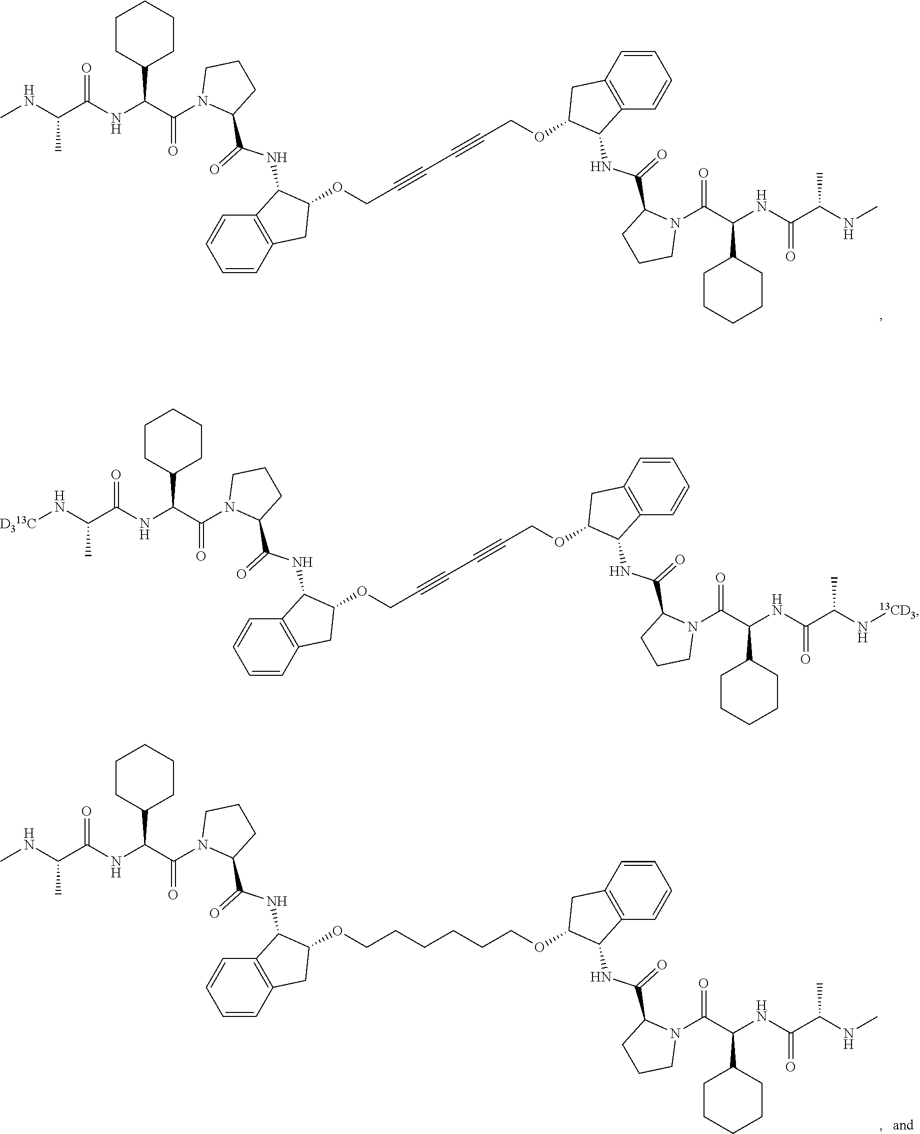

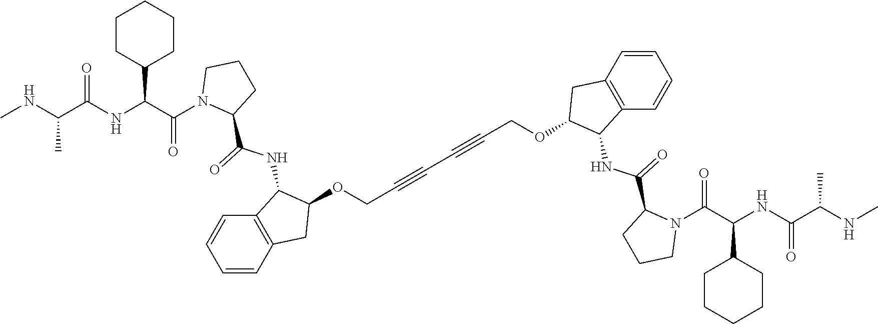

or a pharmaceutically acceptable salt thereof.

In certain embodiments, the IAP Inhibitor agent(s) is a potent antagonist of XIAP and binds to XIAP with a K_{D} of 250 nM or less, more preferably 100 nM, 50 nM, 10 nM or 1 nM or less.

In certain embodiments, the IAP Inhibitor agent(s) is a potent antagonist of XIAP, having an IC₅₀ for XIAP inhibition 250 nM or less, more preferably 100 nM, 50 nM, 10 nM or 1 nM or less.

In certain embodiments, the IAP Inhibitor agent(s) is a potent antagonist of XIAP and cIAP1, and binds to each of XIAP and cIAP1 with K_{D}'s of 250 nM or less, more preferably 100 nM, 50 nM, 10 nM or 1 nM or less.

In certain embodiments, the IAP Inhibitor agent(s) is a potent antagonist of XIAP and cIAP1, having an IC₅₀ for each of XIAP inhibition and cIAP1 inhibition of 250 nM or less, more preferably 100 nM, 50 nM, 10 nM or 1 nM or less.

In some embodiments, the present disclosure provides a compound of Formula I, or a pharmaceutically acceptable salt thereof, wherein the compound has a XIAP K_{D} of ≤250 nM. In certain embodiments the compound of Formula I has a XIAP K_{D} of ≤100 nM, ≤50 nM, ≤10 nM, or ≤1 nM.

In some embodiments, the present disclosure provides a compound of Formula I, or a pharmaceutically acceptable salt thereof, wherein the compound has a cIAP1 K_{D} of ≤250 nM. In certain embodiments the compound of Formula I has a cIAP1 K_{D} of ≤100 nM, ≤50 nM, ≤10 nM, or ≤1 nM. In some embodiments, the present disclosure provides a compound of Formula I, or a pharmaceutically acceptable salt thereof, wherein the compound has a XIAP K_{D} of ≤250 nM. In certain embodiments the compound of Formula I has a XIAP K_{D} of ≤100 nM, ≤50 nM, ≤10 nM, or ≤1 nM, and a cIAP1 K_{D} of ≤100 nM, ≤50 nM, ≤10 nM, or ≤1 nM.

In certain embodiments, the IAP Inhibitor agent(s) is selected from the group consisting of LCL161 Inhibitor, AZD5582, SM-164, BV6, Xevinapant, GDC-0152, ASTX660, CUDC-427, Embelin (or Embelic acid), MX69, MV1, Polygalacin D, UC-112, HY-125378m Tolinapant (ASTX660) and SBP-0636457, or a pharmaceutically acceptable salt thereof.

In certain embodiments, the IAP inhibitor is a selective XIAP inhibitor (having an IC₅₀ for XIAP inhibition at least 10-fold less than the IC₅₀ for CIAP inhibition, and more preferably at least 20, 50 or 100-fold less), such as SM-164.

In certain embodiments, the formulations of the present disclosure further include at least one ESO Regenerative agent dispersed therein or thereon, wherein the formulation delivers both the IAP Inhibitor agent and ESO Regenerative agent into esophageal tissue.

In certain embodiments, bioadhesive nanoparticle further includes at least one ESO Regenerative agent dispersed therein or thereon, wherein the nanoparticle elutes the both the IAP Inhibitor agent and ESO Regenerative agent into the mucous gel layer when adhered to mucosal tissue.

In certain embodiments, the bioadhesive nanoparticle further includes at least one ESO Regenerative agent dispersed therein or thereon, wherein the nanoparticle elutes the both the IAP Inhibitor agent and ESO Regenerative agent into the mucous gel layer when adhered to mucosal tissue.

In certain embodiments, the ESO Regenerative agent is pan-inhibitor of ABL kinase inhibitor, preferably a BCR-ABL kinase inhibitor. Exemplary pan-inhibitor include imatinib, nilotinib, dasatinib, bosutinib and ponatinib, and is preferably ponatinib.

In certain embodiments, the ESO Regenerative agent is a BACE inhibitor, an FAK inhibitor, a VEGR inhibitor or an AKT inhibitor.

In certain embodiments, the submucosal retentive formulation produces a systemic concentration of the ESO Regenerative Agent, such as ponatinib, which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In still other embodiments, there is provided a submucosal retentive formulation comprising at least one IAP Inhibitor agent and one or more pharmaceutically acceptable excipients, which formulation is injectable submucosally and forms a submucosal depot releasing an effective amount of the IAP Inhibitor agent into the surrounding tissue.

In certain embodiments, the submucosal retentive formulation is an injectable thermogel for submucosal injection, comprising at least one IAP Inhibitor agent and one or more pharmaceutically acceptable excipients, wherein the thermogel has a low-viscosity fluid at room temperature (and easily injected), and becomes a non-flowing gel at body temperature after injection.

In certain embodiments, the submucosal retentive formulations further include at least one ESO Regenerative agent dispersed therein, wherein the submucosal retentive formulations release the both the IAP Inhibitor agent and ESO Regenerative agent into the tissue surrounding the site of submucosal injection.

In certain embodiments, the ESO Regenerative agent is TAK1 inhibitor. Exemplary TAK1 inhibitors include 5Z-7-oxozeaenol, dehydroabietic acid, NG25, sarsasapogenin, takinib, TAK1-IN1, minnelide and triptolide, or a pharmaceutically acceptable salt or mixture thereof.

In certain embodiments, the ESO Regenerative agent is a RET inhibitor.

In some embodiments, the RET inhibitor is a compound of Formula II:

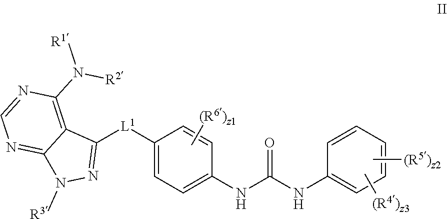

or a pharmaceutically acceptable salt thereof, wherein:

R^{1′} and R^{2′} are independently hydrogen or substituted or unsubstituted alkyl;

R^{3′} is substituted or unsubstituted alkyl;

each R^{4′} is independently hydrogen, halogen, —C(X)₃, —CN, —OH, —COOH, —CONH₂, —NO, —NO₂, —C(O)H, —SH, —SO₂Cl, —SO₃H, —SO₄H, —SO₂NH₂, —NHNH₂, —ONH₂, —NHC═(O)NHNH₂, —C(O)CH₃, —NHC═(O)NH₂, —NHSO₂H, —NHC═(O)H, —NHC(O)OH, —NHOH, —OCF₃, —OCHF₂, or substituted or unsubstituted alkyl;

each R^{5′} is independently halogen, —CN, —C(X^{a})₃, —S(O)₂H, —NO, —NO₂, —C(O)H, —C(O)NH₂, —S(O)₂NH₂, —OH, —SH, —SO₂Cl, —SO₃H, —SO₄H, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, —CO₂H, or substituted or unsubstituted (C₁-C₆) alkyl;

each R^{6′} is independently halogen, —CN, —C(X^{b})₃, —S(O)₂H, —NO, —NO₂, —C(O)H, —C(O)NH₂, —S(O)₂NH₂, —OH, —SH, —SO₂Cl, —SO₃H, —SO₄H, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, or —CO₂H;

L¹ is independently a bond or substituted or unsubstituted alkylene;

z¹ is an integer from 0 to 4;

z² is an integer from 0 to 5;

z³ is an integer from 0 to 4; and

each of X, X^{a} and X^{b} are independently —F, —Cl, —Br, or —I.

A “substituted” alkyl or alkylene may be substituted with a group selected from —OR′, ═O, ═NR′, ═N—OR′, —NR′R″, —SW, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NW′, —S(O)R′, —S(O)₂R′, —S(O)₂NR′R″, —NRSO₂R′, —NR′NR″R′″, —ONR′R″, —NR′C═(O)NR″NR′″R″″, —CN, and —NO₂, wherein each R, R′, R″, R′″, and R″″ is independently hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups.

A “substituted” aryl and heteroaryl may be substituted with a group selected from —OR′, —NR′R″, —SW, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, and —NO₂, —R′, —N₃, —CH(Ph)₂, fluoro(C₁-C₄)alkoxy, and fluoro(C₁-C₄)alkyl.

In some embodiments, the present disclosure provides a compound of Formula IIa:

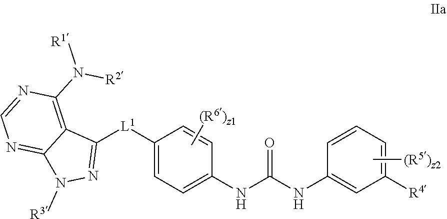

or a pharmaceutically acceptable salt thereof, wherein each of R^{1′}, R^{2′}, R^{3′}, R^{4′}, R^{5′}, R^{6′}, L¹, z¹, and z² is as defined above and described herein.

In some embodiments, the present disclosure provides a compound of Formula IIb:

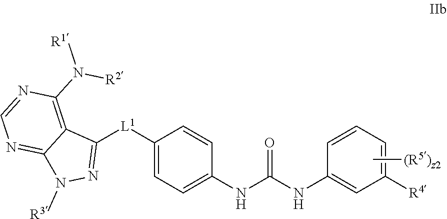

or a pharmaceutically acceptable salt thereof, wherein each of R^{1′}, R^{2′}, R^{3′}, R^{4′}, R^{5′}, L¹, and z² is as defined above and described herein.

In some embodiments, the present disclosure provides a compound of Formula IIc:

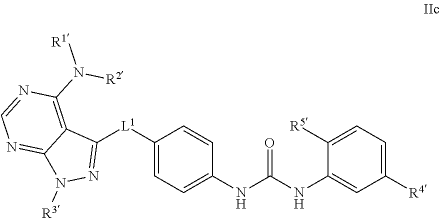

or a pharmaceutically acceptable salt thereof, wherein each of R^{1′}, R^{2′}, R^{3′}, R^{4′}, R^{5′}, L¹, and z² is as defined above and described herein.

In some embodiments, R^{1′} is hydrogen. In some embodiments, R^{1′} is substituted or unsubstituted alkyl. In some embodiments, R^{1′} is unsubstituted alkyl. In some embodiments, R^{1′} is unsubstituted (C₁-C₆) alkyl. In some embodiments, R^{1′} is unsubstituted (C₁-C₄) alkyl. In some embodiments, R^{1′} is methyl. In some embodiments, R^{1′} is ethyl. In some embodiments, R^{1′} is n-propyl. In some embodiments, R^{1′} is isopropyl. In some embodiments, R^{1′} is n-butyl. In some embodiments, R^{1′} is t-butyl. In some embodiments, R^{1′} is n-pentyl. In some embodiments, R^{1′} is substituted alkyl. In some embodiments, R^{1′} is substituted (C₁-C₆) alkyl. In some embodiments, R^{1′} is substituted (C₁-C₄) alkyl.

In some embodiments, R^{2′} is hydrogen. In some embodiments, R^{2′} is substituted or unsubstituted alkyl. In some embodiments, R^{2′} is unsubstituted alkyl. In some embodiments, R^{2′} is unsubstituted (C₁-C₆) alkyl. In some embodiments, R^{2′} is unsubstituted (C₁-C₄) alkyl. In some embodiments, R^{2′} is methyl. In some embodiments, R^{2′} is ethyl. In some embodiments, R^{2′} is n-propyl. In some embodiments, R^{2′} is isopropyl. In some embodiments, R^{2′} is n-butyl. In some embodiments, R^{2′} is t-butyl. In some embodiments, R^{2′} is n-pentyl. In some embodiments, R^{2′} is substituted alkyl. In some embodiments, R^{2′} is substituted (C₁-C₆) alkyl. In some embodiments, R^{2′} is substituted (C₁-C₄)alkyl.

In some embodiments, L¹ is a bond. In some embodiments, L¹ is substituted or unsubstituted alkylene. In some embodiments, L¹ is unsubstituted alkylene. In some embodiments, L¹ is unsubstituted (C₁-C₆)alkylene. In some embodiments, L¹ is unsubstituted (C₁-C₄)alkylene. In some embodiments, L¹ is methylene. In some embodiments, L¹ is ethylene. In some embodiments, L¹ is n-propylene. In some embodiments, L¹ is isopropylene. In some embodiments, L¹ is n-butylene. In some embodiments, L¹ is t-butylene. In some embodiments, L¹ is n-pentylene. In some embodiments, L¹ is substituted alkylene. In some embodiments, L¹ is substituted (C₁-C₆) alkylene. In some embodiments, L¹ is substituted (C₁-C₄) alkylene.

In some embodiments, R^{3′} is substituted or unsubstituted alkyl. In some embodiments, R^{3′} is unsubstituted alkyl. In some embodiments, R^{3′} is unsubstituted (C₁-C₆) alkyl. In some embodiments, R^{3′} is unsubstituted (C₁-C₄) alkyl. In some embodiments, R^{3′} is methyl. In some embodiments, R^{3′} is ethyl. In some embodiments, R^{3′} is n-propyl. In some embodiments, R^{3′} is isopropyl. In some embodiments, R^{3′} is n-butyl. In some embodiments, R^{3′} is t-butyl. In some embodiments, R^{3′} is n-pentyl. In some embodiments, R^{3′} is substituted alkyl. In some embodiments, R^{3′} is substituted (C₁-C₆) alkyl. In some embodiments, R^{3′} is substituted (C₁-C₄) alkyl.

In some embodiments, R^{4′} is independently hydrogen, halogen, —C(X)₃, —CN, —OH, —COOH, —CONH₂, —NO, —NO₂, —C(O)H, —SH, —SO₂Cl, —SO₃H, —SO₄H, —SO₂NH₂, —NHNH₂, —ONH₂, —NHC═(O)NHNH₂, —C(O)CH₃, —NHC═(O)NH₂, —NHSO₂H, —NHC═(O)H, —NHC(O)—OH, —NHOH, —OCF₃, —OCHF₂, or substituted or unsubstituted alkyl. In some embodiments, R^{4′} is independently halogen, —CN, —C(X)₃, —NO, —NO₂, —C(O)H, or —CO₂H. In some embodiments, R^{4′} is halogen. In some embodiments, R^{4′} is —CN. In some embodiments, R^{4′} is —NO. In some embodiments, R^{4′} is —NO₂. In some embodiments, R^{4′} is —C(O)H. In some embodiments, R^{4′} is —CO₂H. In some embodiments, R^{4′} is halogen or —C(X)₃. In some embodiments, R^{4′} is —C(X)₃. In some embodiments, X is —F. Accordingly, in some embodiments, R^{4′} is, e.g., —CF₃. In some embodiments, X is —Cl. In some embodiments, X is —Br. In some embodiments, X is —I. In some embodiments, R^{4′} is —F. In some embodiments, R^{4′} is —Cl. In some embodiments, R^{4′} is —Br. In some embodiments, R^{4′} is —I. In some embodiments, R^{4′} is substituted or unsubstituted alkyl. In some embodiments, R^{4′} is unsubstituted alkyl. In some embodiments, R^{4′} is unsubstituted (C₁-C₆) alkyl. In some embodiments, R^{4′} is unsubstituted (C₁-C₄) alkyl. In some embodiments, R^{4′} is methyl. In some embodiments, R^{4′} is ethyl. In some embodiments, R^{4′} is n-propyl. In some embodiments, R^{4′} is isopropyl. In some embodiments, R^{4′} is n-butyl. In some embodiments, R^{4′} is t-butyl. In some embodiments, R^{4′} is n-pentyl. In some embodiments, R^{4′} is substituted alkyl. In some embodiments, R^{4′} is substituted (C₁-C₆) alkyl. In some embodiments, R^{4′} is substituted (C₁-C₄) alkyl.

In some embodiments, R^{5′} is independently hydrogen, halogen, —C(X^{a})₃, —CN, —OH, —COOH, —CONH₂, —NO, —NO₂, —C(O)H, —SH, —SO₂Cl, —SO₃H, —SO₄H, —SO₂NH₂, —NHNH₂, —ONH₂, —NHC═(O)NHNH₂, —C(O)CH₃, —NHC═(O)NH₂, —NHSO₂H, —NHC═(O)H, —NHC(O)—OH, —NHOH, —OCF₃, —OCHF₂, or substituted or unsubstituted alkyl. In some embodiments, R^{5′} is independently halogen, —CN, —C(X^{a})₃, —NO, —NO₂, —C(O)H, or —CO₂H. In some embodiments, R^{5′} is halogen. In some embodiments, R^{5′} is —CN. In some embodiments, R^{5′} is —NO. In some embodiments, R^{5′} is —NO₂. In some embodiments, R^{5′} is —C(O)H. In some embodiments, R^{5′} is —CO₂H. In some embodiments, R^{5′} is halogen or —C(X^{a})₃. In some embodiments, R^{5′} is —C(X^{a})₃. In some embodiments, X^{a} is —F (i.e. R^{5′} is —CF₃). In some embodiments, X^{a} is —Cl. In some embodiments, X^{a} is —Br. In some embodiments, X^{a} is —I. In some embodiments, R^{5′} is —F. In some embodiments, R^{5′} is —Cl. In some embodiments, R^{5′} is —Br. In some embodiments, R^{5′} is —I. In some embodiments, R^{5′} is substituted or unsubstituted alkyl. In some embodiments, R^{5′} is unsubstituted alkyl. In some embodiments, R^{5′} is unsubstituted (C₁-C₆)alkyl. In some embodiments, R^{5′} is unsubstituted (C₁-C₄)alkyl. In some embodiments, R^{5′} is methyl. In some embodiments, R^{5′} is ethyl. In some embodiments, R^{5′} is n-propyl. In some embodiments, R^{5′} is isopropyl. In some embodiments, R^{5′} is n-butyl. In some embodiments, R^{5′} is t-butyl. In some embodiments, R^{5′} is n-pentyl. In some embodiments, R^{5′} is substituted alkyl. In some embodiments, R^{5′} is substituted (C₁-C₆)alkyl. In some embodiments, R^{5′} is substituted (C₁-C₄)alkyl.

In some embodiments, R^{6′} is independently hydrogen, halogen, —C(X^{b})₃, —CN, —OH, —COOH, —CONH₂, —NO, —NO₂, —C(O)H, —SH, —SO₂Cl, —SO₃H, —SO₄H, —SO₂NH₂, —NHNH₂, —ONH₂, —NHC═(O)NHNH₂, —C(O)CH₃, —NHC═(O)NH₂, —NHSO₂H, —NHC═(O)H, —NHC(O)—OH, —NHOH, —OCF₃, or —OCHF₂. In some embodiments, R^{6′} is halogen, —CN, —C(X^{b})₃, —NO, —NO₂, —C(O)H, or —CO₂H. In some embodiments, R^{6′} is halogen. In some embodiments, R^{6′} is —CN. In some embodiments, R^{6′} is —NO. In some embodiments, R^{6′} is —NO₂. In some embodiments, R^{6′} is —C(O)H. In some embodiments, R^{6′} is —CO₂H. In some embodiments, R^{6′} is halogen or —C(X^{b})₃. In some embodiments, R^{6′} is —C(X^{b})₃. In some embodiments, X^{b} is —F (i.e. R^{6′} is —CF₃). In some embodiments, X^{b} is —Cl. In some embodiments, X^{b} is —Br. In some embodiments, X^{b} is —I. In some embodiments, R^{6′} is —F. In some embodiments, R^{6′} is —Cl. In some embodiments, R^{6′} is —Br. In some embodiments, R^{6′} is —I.

In some embodiments, z¹ is 1 to 4. In some embodiments, z¹ is 1 to 3. In some embodiments, z¹ is 1 to 2. In some embodiments, z¹ is 0 to 4. In some embodiments, z¹ is 0 to 3. In some embodiments, z¹ is 0 to 2. In some embodiments, z¹ is 0 to 1. In some embodiments, z¹ is 0. In some embodiments, z¹ is 1. In some embodiments, z¹ is 2. In some embodiments, z¹ is 3. In some embodiments, z¹ is 4.

In some embodiments, z² is 1 to 5. In some embodiments, z² is 1 to 4. In some embodiments, z² is 1 to 3. In some embodiments, z² is 1 to 2. In some embodiments, z² is 0 to 5. In some embodiments, z² is 0 to 4. In some embodiments, z² is 0 to 3. In some embodiments, z² is 0 to 2. In some embodiments, z² is 0 to 1. In some embodiments, z² is 0. In some embodiments, z² is 1. In some embodiments, z² is 2. In some embodiments, z² is 3. In some embodiments, z² is 4. In some embodiments, z² is 5.

In some embodiments, z³ is 1 to 4. In some embodiments, z³ is 1 to 3. In some embodiments, z³ is 1 to 2. In some embodiments, z³ is 0 to 4. In some embodiments, z³ is 0 to 3. In some embodiments, z³ is 0 to 2. In some embodiments, z³ is 0 to 1. In some embodiments, z³ is 0. In some embodiments, z³ is 1. In some embodiments, z³ is 2. In some embodiments, z³ is 3. In some embodiments, z³ is 4.

In some embodiments, R^{4′} is CF₃. In some embodiments, R^{5′} is halogen. In some embodiments, each R^{1′} and R^{2′} is hydrogen. In some embodiments, L¹ is a bond. In some embodiments, R^{3′} is unsubstituted alkyl (e.g., C₁-C₆ alkyl). In some embodiments, a compound of Formula II is selected from

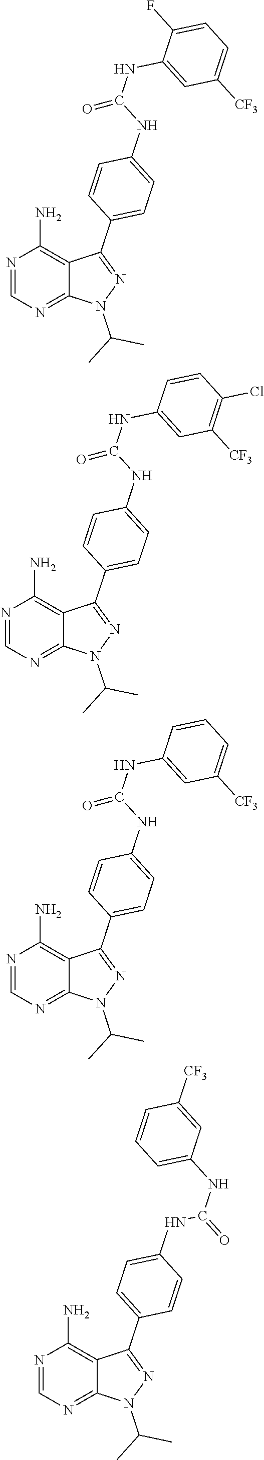

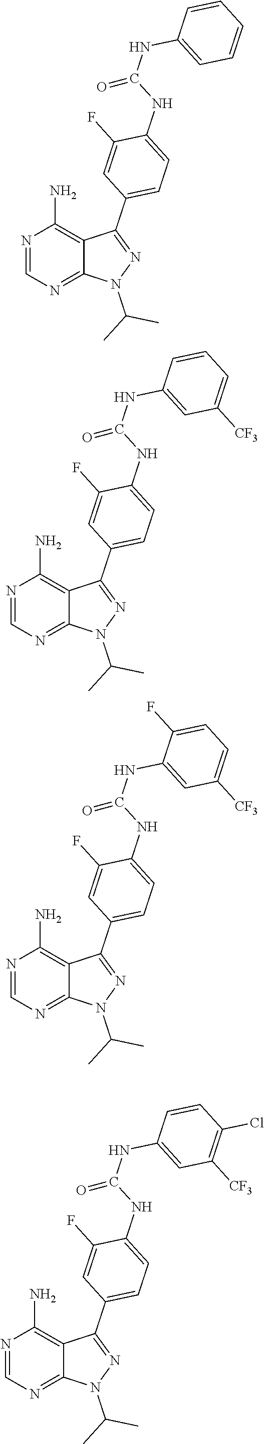

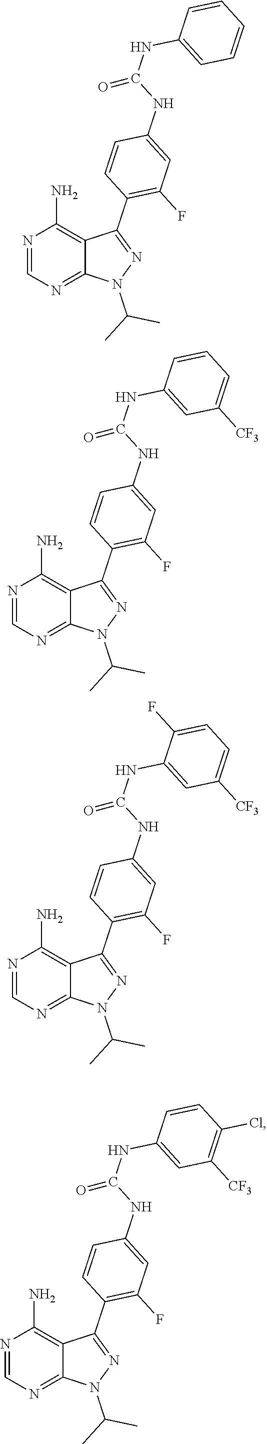

or a pharmaceutically acceptable salt thereof.

Exemplary RET inhibitors include AD80, Regorafenib (BAY 73-4506), Cabozantinib malate (XL184), Fedratinib (TG101348), Danusertib (PHA-739358), TG101209, Agerafenib (RXDX-105), Regorafenib Hydrochloride, Selpercatinib (LOXO-292), Pralsetinib (BLU-667), GSK3179106, Regorafenib (BAY-734506) Monohydrate, vandetanib, RXDX-105, lenvatinib, sorafenib, sunitinib, dovitinib, alectinib, ponatinib, regorafenib, nintedanib, apatinib, motesanib, BLU-667, and LOXO-292, or a pharmaceutically acceptable salt or mixture thereof.

In certain embodiments, the ESO Regenerative agent is pan-inhibitor of ABL kinase inhibitor, preferably a BCR-ABL kinase inhibitor. Exemplary pan-inhibitor include imatinib, nilotinib, dasatinib, bosutinib and ponatinib, and is preferably ponatinib.

In certain embodiments, the ESO Regenerative agent is a BACE inhibitor, an FAK inhibitor, a VEGR inhibitor or an AKT inhibitor.

For instance, the submucosal retentive formulation can have a submucosal residence half-life in esophageal tissue of at least 30 minutes, more preferably at least 60, 120, 180, 240 or even 300 minutes.

For instance, the submucosal retentive formulation can produce at least a minimally effective concentration (MEC) of the IAP Inhibitor agent in the esophageal tissue in which it is injected for at least 30 minutes, more preferably at least 60, 120, 180, 240 or even 300 minutes.

For instance, the submucosal retentive formulation can produce IAP Inhibitor agent concentration in esophageal tissue in which it is injected with T1/2 of at least 2 hours, more preferably at least 4, 6, 8, 10 or even 12 hours.

The present disclosure also provides submucosal retentive formulations which further include one or more ESO Regenerative Agents in addition to the IAP Inhibitor agent(s). For example the formulation can include (i) a BCR-ABL kinase inhibitor, and (ii) one or more pharmaceutically acceptable excipients, which formulation is injectable submucosally and forms a submucosal depot releasing an effective amount of the BCR-ABL kinase inhibitor to the surrounding tissue. In certain preferred embodiments, the BCR-ABL kinase inhibitor is ponatinib. In certain preferred embodiments, the BCR-ABL kinase inhibitor is a FLT3 inhibitor such as quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

In certain embodiments, the submucosal retentive formulation can also produce at least a minimally effective concentration (MEC) of the ESO Regenerative Agent in the esophageal tissue in which it is injected for at least 30 minutes, more preferably at least 60, 120, 180, 240 or even 300 minutes.

In certain embodiments, the submucosal retentive formulation can also produce an ESO Regenerative Agent concentration in esophageal tissue in which it is injected with T1/2 of at least 2 hours, more preferably at least 4, 6, 8, 10 or even 12 hours.

In certain embodiments, the submucosal retentive formulation produces a systemic concentration of the ESO Regenerative Agent, such as ponatinib, which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In each of the above submucosal retentive formulations, the formulation can form a flowable and/or viscous gel.

In certain embodiments, the formulation is an injectable thermogel. Thermogels includes, merely to illustrate, poly(lactic acid-co-glycolic acid)-poly(ethylene glycol)-poly(lactic acid-co-glycolic acid) (PLGA-PEG-PLGA) triblock copolymers.

In certain embodiments, the formulation is a hydrogel.

In certain embodiments, the formulation is suitable for endoscopic dissection.

In certain embodiments, the formulation further comprises an anticoagulant.

In certain embodiments, the formulation further comprises one or more antitussives, antihistamines, antipyretics, analgesics, anti-infective agents and/or chemotherapeutic agents

Another aspect of the present disclosure provides an injectable thermogel for submucosal injection, comprising an IAP Inhibitor agent (such as SM-164) and ponatinib and (optionally) one or more pharmaceutically acceptable excipients, wherein the thermogel has a low-viscosity fluid at room temperature (and easily injected), and becomes a non-flowing gel at body temperature after injection.

In certain embodiments, the disclosure provides an esophageal topical retentive formulation for topical application to the luminal surface of the esophagus, comprising (i) an IAP Inhibitor agent and (optionally) an ESO Regenerative Agent, (ii) a bioadhesive, and (iii) optionally, one or more pharmaceutically acceptable excipients.

For example the formulation can include an IAP inhibitor which is a selective XIAP inhibitor (having an IC₅₀ for XIAP inhibition at least 10-fold less than the IC₅₀ for CIAP inhibition, and more preferably at least 20, 50 or 100-fold less), such as SM-164.

Where the formulation includes a ESO Regenerative Agent, such agents include (i) a BCR-ABL kinase inhibitor, and (ii) one or more pharmaceutically acceptable excipients, which formulation is injectable submucosally and forms a submucosal depot releasing an effective amount of the BCR-ABL kinase inhibitor to the surrounding tissue. In certain preferred embodiments, the BCR-ABL kinase inhibitor is ponatinib. In certain preferred embodiments, the BCR-ABL kinase inhibitor is a FLT3 inhibitor such as quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

In certain embodiments, the topical formulation is a viscous bioadhesive liquid to coat the esophagus.

In certain embodiments, the topical formulation comprises IAP Inhibitor agent eluting multiparticulates, microparticles, nanoparticles or microdiscs

In certain embodiments, the topical formulation further comprises an anticoagulant.

In certain embodiments, the topical formulation further comprises one or more antitussives, antihistamines, antipyretics, analgesics, anti-infective agents and/or chemotherapeutic agents

In further embodiments, there is provided bioadhesive nanoparticle having a polymeric surface with an adhesive force equivalent to an adhesive force of between 10 N/m² and 100,000 N/m² measured on human mucosal surfaces, which nanoparticle further includes at least one IAP Inhibitor agent, the IAP Inhibitor agent dispersed therein or thereon, wherein the nanoparticle elutes the IAP Inhibitor agent into the mucous gel layer when adhered to mucosal tissue.

For example the formulation can include (i) an IAP inhibitor, such as SM-164, and (ii) one or more pharmaceutically acceptable excipients, which formulation is injectable submucosally and forms a submucosal depot releasing an effective amount of the IAP Inhibitor agent inhibitor to the surrounding tissue. In certain preferred embodiments, the formulation also includes a BCR-ABL kinase inhibitor, such as ponatinib.

In certain embodiments, the submucosal retentive formulation produces a systemic concentration of the IAP Inhibitor agent, such as SM-164, which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In certain embodiments, the bioadhesive nanoparticle further comprises an anticoagulant.

In certain embodiments, the bioadhesive nanoparticle further comprises one or more antitussives, antihistamines, antipyretics, analgesics, anti-infective agents and/or chemotherapeutic agents

In further embodiments, there is provided a drug eluting device, which device comprises drug release means including an IAP Inhibitor agent, which device when deployed in a patient positions the drug release means proximal to target esophageal tissue and releases the agent in an amount sufficient to achieve a therapeutically effective exposure of the target esophageal tissue.

For instance, the drug eluting device can produce at least a minimally effective concentration (MEC) of the IAP Inhibitor agent in the target esophageal tissue to which it is applied to which it is applied for at least 30 minutes, more preferably at least 60, 120, 180, 240 or even 300 minutes.

For instance, the drug eluting device can produce IAP Inhibitor agent concentration in the esophageal tissue to which it is applied with T1/2 of at least 2 hours, more preferably at least 4, 6, 8, 10 or even 12 hours.

In certain embodiments, the drug eluting device produces a systemic concentration of the IAP Inhibitor agent which is less than ⅓^{rd} the maximum tolerated does (MTD) for that agent, and even more preferably less than ⅕^{th}, 1/10^{th}, 1/20^{th}, 1/50^{th} or even 1/100^{th} the maximum tolerated does (MTD) for that agent.

In certain embodiments, the drug eluting device is for treating one or more of esophagitis, Barrett's esophagus, esophageal dysplasia or esophageal cancer, which device comprises drug release means including an Anti-BESC Agent that selectively kills or inhibits the proliferation or differentiation of Barrett's Esophagus stem cells (BESC) relative to normal esophageal stem cells, which device when deployed in a patient positions the drug release means proximal to the luminal surface of the esophagus and releases the agent in an amount sufficient to achieve a therapeutically effective exposure of the luminal surface to the agent.

Exemplary drug eluting devices include biodegradable stents, self-expandable stents, such as a self-expandable metallic stent (SEMS) or self-expandable plastic stent (SEPS), chips and wafers for submucosal implantation, and the like.

In other embodiments, the drug eluting device is a device for extraluminal placement, such as a microneedle cuff.

In certain embodiments, the IAP Inhibitor agent is co-administered with an analgesic, and an anti-infective or both. These may be administered as separate formulation, or optionally, may be the IAP Inhibitor agent is co-formulated with the analgesic or the anti-infective or both.

In certain embodiments, the IAP Inhibitor agent is formulated as a liquid for oral delivery to the esophagus.

In certain embodiments, the IAP Inhibitor agent is formulated as a single oral dose.

In certain embodiments, the IAP Inhibitor agent is delivered by a drug eluting device that is a drug eluting stent.

In certain embodiments, the IAP Inhibitor agent is delivered by a drug eluting device that is a balloon catheter having a surface coating including the agent.

In certain embodiments, the IAP Inhibitor agent is cell permeable, such as characterized by a permeability coefficient of 10⁻⁹ or greater, more preferably 10⁻⁸ or greater or 10⁻⁷ or greater.

One aspect of the disclosure provides a single oral dosage formulation comprising (i) an IAP Inhibitor agent, (ii) an ESO Regenerative Agent, and (iii) and a pharmaceutically acceptable excipient, which single oral dosage formulation taken by an adult human patient produces a concentration of IAP Inhibitor agent and ESO Regenerative Agent in esophageal tissue effective to slow or reverse the progress of an esophageal metaplasia, dysplasia, cancer or a combination thereof. In certain preferred embodiments, the BCR-ABL kinase inhibitor is ponatinib. In certain preferred embodiments, the BCR-ABL kinase inhibitor is a FLT3 inhibitor such as quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

In certain embodiments, the methods, preparations and devices of the present disclosure are intended (and appropriate) for use in human patients.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating certain embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1a-f. Clonogenic cells of patient-matched lesions in EAC. FIG. 1a. White-light imaging of distal esophagus depicting biopsy sites of co-existing mucosal lesions. EAC, esophageal adenocarcinoma; DYS, dysplasia; BE, Barrett's; ESO, normal esophagus. FIG. 1b. Generation of single cell derived library of clonogenic cells from colony-forming cells of indicated biopsy. FIG. 1c. Phase-contrast image of colonies derived from single cell clones. FIG. 1d. Immunofluorescence micrograph of section of epithelia from air-liquid interface (ALI) differentiation of discrete clones of BE1, BE2, Dysplasia, and EAC showing distribution of antibodies to E-cadherin (red) and Ki67 (green). FIG. 1e. Histological sections of nodules resulting from xenografting of stem cells of BE1, BE2, DYS, and EAC clones in immunodeficient mice. FIG. 1f. Graphical representation of nodule growth following stem cell xenografting to immunodeficient mice. Error bars, SD.

FIGS. 2a-i. Clone variation and genomic stability of lesional stem cells. FIG. 2a. Copy number variation (CNV) profiles of clones sampled from indicated biopsy libraries determined from low-pass whole genome sequencing. CN, copy number. FIG. 2b. CNV profiles of selected clones determine by exome sequencing. FIG. 2c. Histogram of allele frequency distribution for all somatic single nucleotide mutations across 35 clones from Case 1. FIG. 2d. Percentage overlap of SNV events among EAC clones derived from a single 1 mm biopsy. FIG. 2e. Copy ratio profile of chromothripsis event on chromosome 16 in single dysplasia and EAC clones. FIG. 2f. Schematic for analysis of genetic stability of EAC clone through serial passaging in vitro and after tumor formation in mice. FIGS. 2g-h. Copy ratio profile of EAC clone C1D1-7 determined from whole exome sequencing. FIG. 2i. Copy number variation profiles of EAC clone C1D1-7 following in vitro propagation and xenografting for tumor formation in mice. FIG. 2h. Variant allele fraction profiles of subclones clones presented in FIG. 2i.

FIGS. 3a-e. Genomic progression of patient-matched lesional stem cells to EAC. FIG. 3a. Phylogenetic tree of 34 cloned stem cell lineages based on 445 somatic SNVs. Positions of sustained mutations impacting p16, ERBB2, p53, and other genes are indicated. FIG. 3b. Heatmap reflecting variant allele fraction of the 445 somatic SNVs. FIG. 3c. Heatmap of 40 amplified loci (designated numerically and by single marker gene) across indicated lesional stem cells. Those marked by red are from Chr. 16. FIG. 3d. Heatmap of 40 deleted loci (designated numerically and by single marker gene) across indicated lesional stem cells. FIG. 3e. CNV-mediated deletion status of indicated tumor suppressor genes across the 35 lesional stem cell clones used in the phylogenetics analysis.

FIGS. 4a-e. Genomic progression of patient-matched lesional stem cells in EAC case 2. FIG. 4a. Phylogenetic tree of 44 patient-matched stem cell clones from biopsies of a second EAC case based on 515 somatic SNVs. Positions of sustained mutations impacting p16, ARID1A, ERBB2, p53, and other genes are indicated. CTB, chromothripsis of Chr. 8; GD, genome duplication. FIG. 4b. Heatmap of variant allele fraction of the 515 somatic SNVs. FIG. 4c. CNV profiles of across clones determined from exome sequencing. FIG. 4d. Progression of discrete amplification events across clones from indicated lesions. FIG. 4e. CNV deletion events across clones marked by one included gene in each.

FIGS. 5a-e. Transitions among patient-matched lesions. FIG. 5a. Representation of epithelial transitions from Barrett's to EAC. FIG. 5b. Summary of mutational events in lesions accompanying the evolution of EAC in two cases. FIG. 5c. Schematic representation of mutational events (non-synonymous mutations, stop-gain, indels, CNV events) sustained at each transition to more advanced lesions. FIG. 5d. Principal component analysis of whole genome RNA-seq profiling of ALI-differentiated clones representative of BE1, BE2 (LGD), DYS, and EAC as well as patient-matched, normal ESO. FIG. 5e. Volcano plot of differential gene expression between ALI differentiated clones of BE1 and BE2 from Case 1. Genes highlighted in red are those from amplified loci.

FIGS. 6a-g. Drug development for precursor lesions. FIG. 6a. Representative 384-well plate bearing BE1 stem cells after incubation with compounds from drug libraries with magnified wells depicting effects of neutral and deleterious drugs. FIG. 6b. Two-dimensional plot comparing impact on survival of compounds on BE1 versus normal esophageal (ESO) stem cells highlighting drugs of potential interest (circled). FIG. 6c. Dose-response curves of candidate drug (CEP-18770). FIG. 6d. Histogram of esophageal stem cell survival in response to all 1832 compounds in Selleck bioactive compound library. Micrographs show impact of ponatinib on the growth of ESO colonies. FIG. 6e. Two-dimensional survival plot of drug screen against ESO and BE1 in the presence of ponatinib with highlighting of potential “hits”. FIG. 6f. Upper panel: Dose-response plots of XIAP inhibitor SM-164 against esophageal stem cells (ESO) and BE1 stem cells in the presence and absence of ponatinib. Lower panel: Dose-response curves of SM-163/ponatinib against ESO, BE1, BE2, DYS, and EAC stem cell clones. FIG. 6g. Upper panel: Co-cultures of BE1 (KRT7+, green) and ESO (KRT14+, red) stem cells following 72 hrs in the presence and absence of SM-164 and ponatinib. Lower panel: Co-cultures of BE2/ESO, DYS/ESO, and EAC/ESO stem cells in the absence (top) and presence (bottom) of SM-164/ponatinib. ESO stem cells marked by KRT14 expression (red); neoplastic stem cells by KRT7 (green).

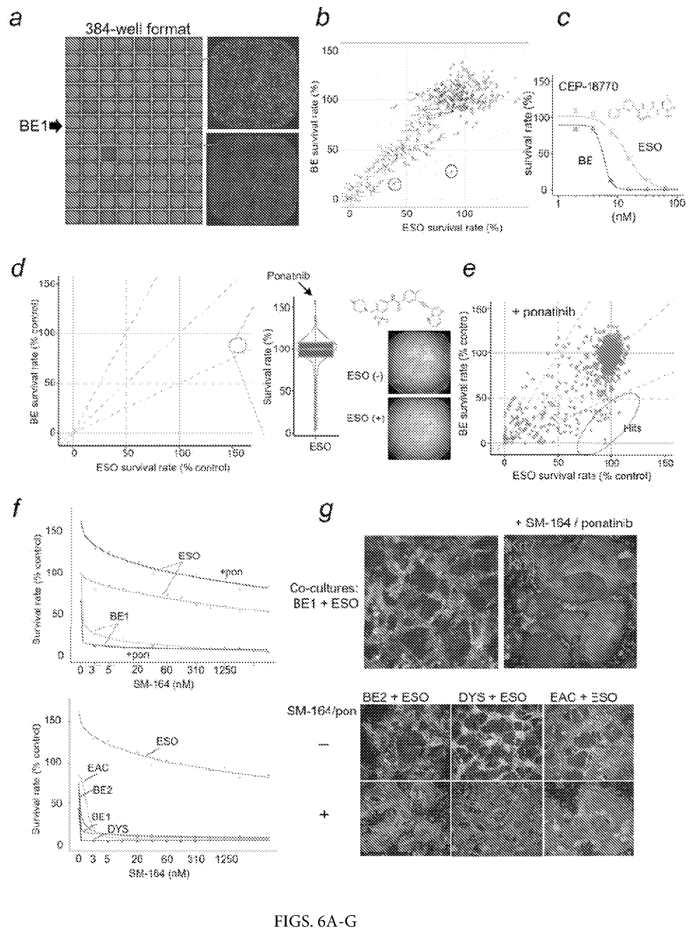

FIG. 7. Is a diagram representing the continuum in certain epithelial tissues of metaplasia to dysplasia to cancer.

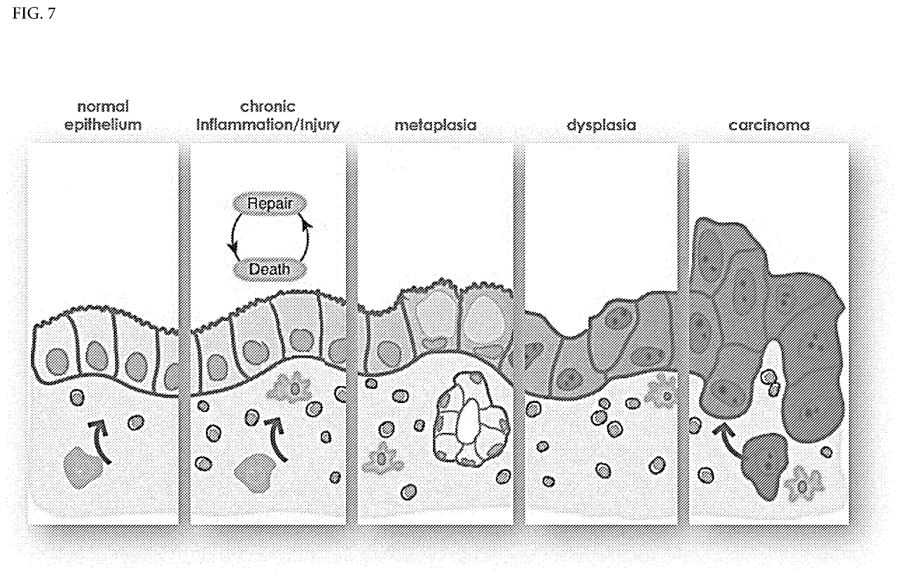

FIG. 8. Is a diagram showing the statistically increasing risk of a patient developing esophageal adenocarcinoma as disease progresses from Barrett's esophagus to high grade dysplasia.

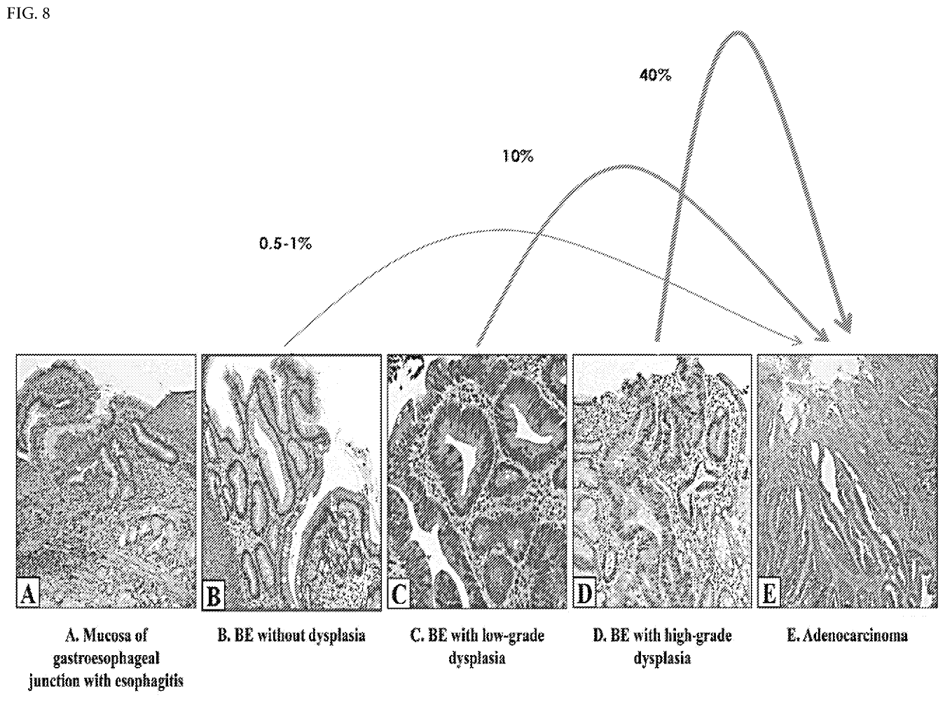

FIGS. 9a-d. In vivo testing in esophageal cancer and gastric cancer. FIG. 9a. Xenograft model of esophageal cancer shows the significant reduction of the tumor size following the ponatinib and SM-164 combination treatment. FIG. 9b. Loss of clonogenicity upon the treatment of ponatinib and SM-164. FIG. 9c. A dramatic reduction of tumor size following treatment of ponatinib and SM-164 in gastric cancer. FIG. 9d. A dramatic reduction of epithelial cancer nodules and associated smooth-muscle actin positive fibrosis following treatment with ponatinib and SM-164 in gastric cancer.

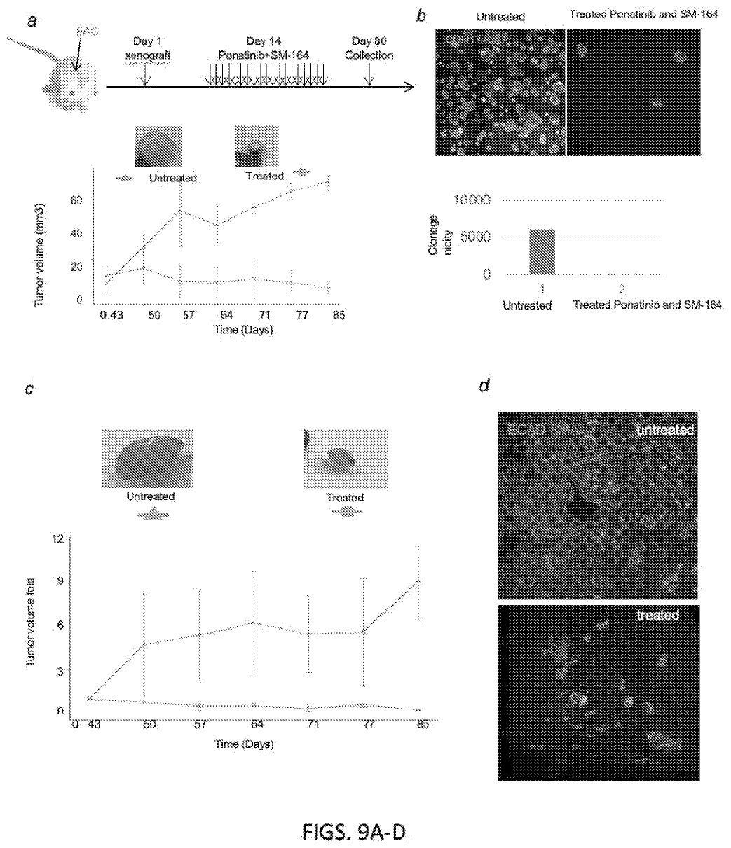

### DETAILED DESCRIPTION

In a recent effort to deconvolute the cellular and genetic heterogeneity of lesional biopsies, the inventors applied technology that enables the cloning of normal gastrointestinal stem cells to endoscopic biopsies of Barrett's esophagus. This work demonstrated that Barrett's esophagus is dependent on a discrete population of highly immature stem cells with immense proliferative potential for its regenerative growth, and that these stem cells differentiate to an intestinal metaplasia indistinguishable from Barrett's esophagus.

### I. Overview

Barrett's Esophagus holds a pivotal position at the interface of cancer biology and patient care. Barrett's was first discovered in 1950's and associated with risk for adenocarcinoma in the 1970's. Barrett's has become a paradigm for precancerous lesions giving rise to progressively more advanced lesions in a process requiring many years supporting an overall escalation model whereby non-cancerous lesions undergo long-term processes of stochastic changes some of which yield more sinister and determinant transitions to low- and high-grade dysplasia which then rapidly and almost inexorably evolve to malignant disease. The recognition of the importance of preemptive therapies that target these premalignant lesions is the foundation of cancer prevention. If true, the clinical solution to preventing the onset of esophageal adenocarcinoma would be simple and direct: ablate Barrett's before it can evolve to more aggressive lesions.

The advance of the development of targeted therapies for Barrett's requires conceptual advance of the origin of Barrett's and the recognition of the existence of Barrett's stem cells. If the premalignant stages of EAC represent the only tractable solution to this disease, it is essential to solve the mystery of the origin of BE and develop new therapeutic strategies specifically targeting its stem cells. However, the ontogeny of BE has been an intriguing puzzle with various hypotheses involving transcommitment of esophageal squamous stem cells, migration from lower gastrointestinal sites, the reparative emergence of submucosal glands, dissemination from bone marrow. The inventors recently showed that BE originated from the opportunistic growth of residual embryonic cells pre-existing at gastroesophageal junction (Wang et al., Cell. 2011 Jun. 24; 145(7):1023-1035). In addition, using the ground state stem cell technology that enabled us to clone stem cells of the normal human gastrointestinal tract, the inventors demonstrated the existence of the stem cells in BE (Yamamoto et al., Nat Commun. 2016 Jan. 19; 7:10380) and suggested they are the key elements to target in a therapeutic program designed to prevent the development and progression of this irreversible and dangerous metaplasia.

In order to uncover drugs specifically targeting BE stem cells that might synergize with physical ablation protocols to further reduce recurrent disease, provided herein is a multiplexed screening of established and experimental drugs or combinations thereof to identify compounds and combinations of compounds that selectively target the particular pathways that dominate the survival of these BE lesions. These BE stem cells were used in hybrid models with normal epithelial squamous stem cells to model the potential ability of such drug combinations to alter the competitive status of such lesions in the distal esophagus.

Also provided herein are screening methods that show the similar selective vulnerabilities of the stem cells of patient-matched BE, dysplasia and EAC, which suggest the broad usage of the pharmacological compositions that would augment physical ablation or mucosal dissection therapies. Indeed, as demonstrated by the data presented herein, the differential sensitivity of the pathogenic stem cells to single agents or combination therapies is carried across multiple tissues and across metaplasia, dysplasia or tumor samples from those tissues.

### II. Definitions

Unless otherwise stated, the following terms used in the specification and claims are defined for the purposes of this Application and have the following meaning:

A “pharmaceutically acceptable salt” of a compound means a salt that is pharmaceutically acceptable and that possesses the desired pharmacological activity of the parent compound. Such salts include: acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference. The compounds of the present disclosure can also exist as cocrystals.

The compounds of the present disclosure may have asymmetric centers. Compounds of the present disclosure containing an asymmetrically substituted atom may be isolated in optically active, racemic forms or other mixtures of isomers. It is well known in the art how to prepare optically active forms, such as by resolution of materials. All chiral, diastereomeric, racemic forms are within the scope of this disclosure, unless the specific stereochemistry or isomeric form is specifically indicated.

Certain compounds of can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Substitution”. As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

“Treating” or “treatment” of a disease includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

### III. Exemplary Embodiments

A. IAP Inhibitor

IAP (Inhibitor of apoptosis) proteins, a family of anti-apoptotic proteins, have an important role in evasion of apoptosis, as they can both block apoptosis-signaling pathways and promote survival. Eight members of this family have been described in humans (BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2). In certain embodiments, the agent is an IAP Inhibitor (i.e., an IAP Antagonist). Exemplary IAP Inhibitors include XIAP inhibitors, CIAP inhibitors, and agents acting as dual XIAP and CIAP inhibitors.

Exemplary IAP inhibitors and antagonists include Birinapant (a bivalent Smac mimetic, which is a potent antagonist for XIAP and cIAP1 with Kds of 45 nM and less than 1 nM, respectively), LCL161 Inhibitor (an IAP inhibitor which inhibits XIAP and cIAP1 with IC₅₀s of 35 and 0.4 nM), AZD5582 (AZD5582 an IAP antagonist which binds to the BIR3 domains cIAP1, cIAP2, and XIAP), SM-164 (a cell-permeable Smac mimetic compound that binds to XIAP protein containing both the BIR2 and BIR3 domains with an IC₅₀ value of 1.39 nM and functions as an extremely potent antagonist of XIAP), BV6 (an antagonist of cIAP1 and XIAP), Xevinapant (or AT-406, is a potent and orally bioavailable Smac mimetic and an antagonist of IAPs, and it binds to XIAP, cIAP1, and cIAP2 proteins), GDC-0152 (a potent IAPs inhibitor, and binds to the BIR3 domains of XIAP, cIAP1, cIAP2 and the BIR domain of ML-IAP), ASTX660 (an orally bioavailable dual antagonist of cIAPs and XIAPs), CUDC-427 (a potent second-generation pan-selective IAP antagonist), Embelin (or Embelic acid, a potent, nonpeptidic XIAP inhibitor). APG-1387 (a bivalent SMAC mimetic and an IAP antagonist, blocks the activity of IAPs family proteins (XIAP, cIAP-1, cIAP-2, and ML-IAP), MX69 (an inhibitor of MDM2/XIAP), AEG40826 (HGS1029) MV1, Polygalacin D, UC-112, AZD5582 dihydrochloride, HY-125378m Tolinapant (ASTX660) and SBP-0636457. In some embodiments, exemplary IAP inhibitors and antagonists include those described in one or more of WO2011098904; WO2009136290; WO2007106192; WO2008014238; WO2008128121 WO2012080271; U.S. Pat. No. 8,202,902; WO2013103703; US20140303090; WO2022130411; WO2017117684 and WO2015092420.

In certain embodiments, the IAP inhibitor is a selective XIAP inhibitor (having an IC₅₀ for XIAP inhibition at least 10-fold less than the IC₅₀ for CIAP inhibition, and more preferably at least 20. 50 or 100-fold less), such as SM-164.

B. Combination Therapies—ESO Regenerative Agent

In certain embodiments, the IAP Inhibitor agent can be administered conjointly with one or more agents that selectively promote proliferation or other regenerative and wound healing activities of normal regenerative esophageal stem cells. Conjoint administration of these “ESO Regenerative agents” may be accomplished by administration of a single co-formulation, by simultaneous administration or by administration at separate times.

In certain embodiments, the IAP Inhibitor agent can be administered conjointly with one or more agents that selectively promote proliferation or other regenerative and wound healing activities of normal esophageal stem cells. Conjoint administration of these “esophageal ESO Regenerative agents” may be accomplished by administration of a single co-formulation, by simultaneous administration or by administration at separate times.

TAK1 Inhibitor. In certain embodiments, the IAP Inhibitor agent is administered conjointly with a TAK1 inhibitor.

“Transforming growth factor activated kinase-1” and “TAK1” are used interchangeably. TAK1 is a protein kinase of the MLK family that mediates signal transduction induced by TGF beta and morphogenetic protein (BMP) and controls a variety of cell functions including transcription regulation and apoptosis. An illustrative non-limitative example of TAK1 is the human TAK1 protein Uniprot database accession number 043318. A “TAK1 inhibitor” as used herein is an agent that reduces or prevents TAK1 activity.

Exemplary embodiments of TAK1 inhibitors include 5Z-7-oxozeaenol, 2-[(aminocarbonyl)amino]-5-[4-(morpholin-4-ylmethyl)phenyl]thiophene-3-carboxamide, 2-[(aminocarbonyl)amino]-5-[4-(1-piperidin-1-ylethyl)phenyl]thiophene-3-carboxamide, 3-[(aminocarbonyl)amino]-5-[4-(morpholin-4-ylmethyl)phenyl]thiophene-2-carboxamide, and 3-[(aminocarbonyl)amino]-5-(4-{[(2-methoxy-2-methylpropyl)amino]methyl}phenyl)thiophene carboxamide.

In still other embodiments, the TAK1 inhibitor is dehydroabietic acid, NG25 (CAS No. 1315355-93-1), sarsasapogenin, takinib, 1-(3-(tert-Butyl)-1-(3-cyanophenyl)-1H-pyrazol-5-yl)-3-(3-methyl-4-(pyridin-4-yloxy)phenyl)urea (PF-05381941 or CAS: 1474022-02-0), 5Z-7-′, TAK1-IN1, minnelide, triptolide or a pharmaceutically acceptable salt or mixture thereof.

In one aspect, provided is a compound according to Formula:

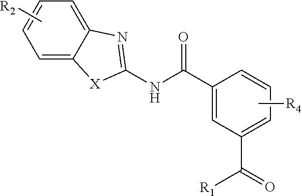

or a stereoisomer or salt thereof;

wherein
X is NR₁ or S;
R₁ is H, C1-4 alkyl, C1-4 carbonyl, or C1-4 carboxyl;
R₂ is H, C1-4 alkyl, C1-4 alkoxy, or halogen;
R₃ is OH, C1-4 alkoxy, or amino; and
R₄ is H, C1-4 alkyl, C1-4 alkoxy, or halogen;

wherein each C1-4 alkyl may be independently substituted by halo, hydroxy, or amino;

In certain embodiments, the TAK1 inhibitor is Takinib, and has the chemical structure

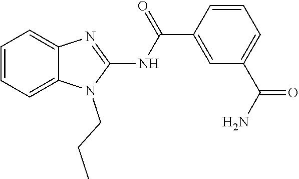 [SMILES: CCCn1c(NC(=O)c2cccc(C(N)=O)c2)nc2ccccc21]

In certain embodiments, the TAK1 inhibitor is NG25, and has the chemical structure

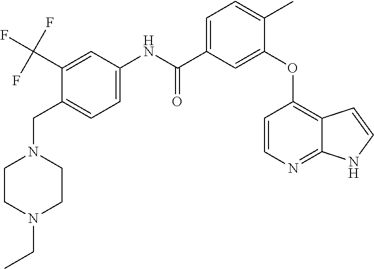 [SMILES: CCN1CCN(Cc2ccc(NC(=O)c3ccc(C)c(Oc4ccnc5[nH]ccc45)c3)cc2C(F)(F)F)CC1]

For example, the TAK1 inhibitor is 5Z-7-Oxozeaenol, having the structure:

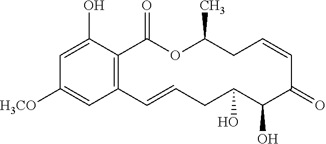 [SMILES: COc1cc(O)c2c(c1)/C=C/C[C@H](O)[C@H](O)C(=O)/C=C\C[C@H](C)OC2=O]

In certain embodiments, the TAK1 inhibitor is an inhibitor of autophosphorylated and non-phosphorylated TAK1 that binds within the ATP-binding pocket and inhibits by slowing down the rate-limiting step of TAK1 activation.

In certain embodiments, the TAK1 inhibitor is an ATP-competitive irreversible inhibitor of TAK1.

In certain embodiments, the TAK1 inhibitor has Ki of 10 μM or less for TAK1 as well as IRAK4, IRAK1, GCK, CLK2, and MINK1.

In certain embodiments, the TAK1 inhibitor has Ki for IRAK4, IRAK1, GCK, CLK2, and MINK1 that is at least 5 times greater than the Ki for TAK1, and even more preferably at least 10, 25, 50 or even 100 times greater.

In certain preferred embodiments, the TAK1 inhibitor has a half maximal inhibitory concentration (IC₅₀) value of 100 nM or less, and even more preferably 50 nM, 25 nM or even 10 nM or less.

In certain embodiments, the TAK1 inhibitor induces TNF-α-dependent induction of apoptosis,

Alternatively, the TAK1 inhibitor is for example an antisense TAK1 nucleic acid, a TAK1 specific short-interfering RNA, or a TAK1-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense TAK1 nucleic acid sequence, an anti-sense TAK1 nucleic acid sequence or both. Optionally, the siRNA is constructed such that a single transcript has both the sense and complementary antisense sequences from the target gene, e.g., a hairpin (shRNA).

c-RET inhibitor. In certain embodiments, the IAP Inhibitor agent is administered conjointly with a RET inhibitor, i.e., an inhibitor or the proto-oncogene tyrosine-protein kinase receptor Ret, also known as Cadherin family member 12 or Proto-oncogene c-Ret; UniprotKB—P07949). For instance, reviews are published disclosing such RET kinase inhibitors (Roskoski et Sadeghi-Nejad, Pharmacol Res. 2018 February; 128:1-17; Zschabitz et Grüllich; Recent Results Cancer Res. 2018; 211:187-198; Grüllich, Recent Results Cancer Res. 2018; 211:67-75; Pitoia et Jerkovich, Drug Des Devel Ther. 2016 Mar. 11; 10:1119-31), the disclosure of which being incorporated herein by reference. Patent applications also disclose RET kinase inhibitors, for instance and non-exhaustively WO18071454, WO18136663, WO18136661, WO18071447, WO18060714, WO18022761, WO18017983, WO17146116, WO17161269, WO17146116, WO17043550, WO17011776, WO17026718, WO14050781, WO07136103, WO06130673, the disclosure of which being incorporated herein by reference.

In certain embodiments, the RET inhibitor is selected from the group consisting of AD80, Regorafenib (BAY 73-4506), Cabozantinib malate (XL184), Fedratinib (TG101348), Danusertib (PHA-739358), TG101209, Agerafenib (RXDX-105), Regorafenib Hydrochloride, Selpercatinib (LOXO-292), Pralsetinib (BLU-667), GSK3179106, Regorafenib (BAY-734506) Monohydrate, vandetanib, RXDX-105, lenvatinib, sorafenib, sunitinib, dovitinib, alectinib, ponatinib, regorafenib, nintedanib, apatinib, motesanib, BLU-667, or LOXO-292.

In certain embodiments, the RET inhibitor may be WHI-P180, Apatinib, CS-2660 (JNJ-38158471), 2-D08,

In certain embodiments, the RET inhibitor is AD80 and has the chemical structure ′

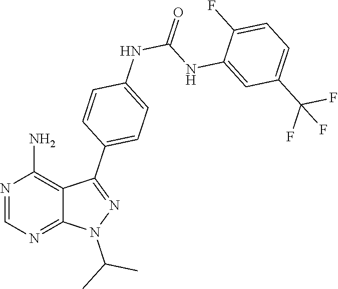 [SMILES: CC(C)n1nc(-c2ccc(NC(=O)Nc3cc(C(F)(F)F)ccc3F)cc2)c2c(N)ncnc21]

In certain embodiments, the RET inhibitor has a half maximal inhibitory concentration (IC₅₀) value of 100 nM or less, and even more preferably 50 nM, 25 nM, 10 nM or even 5 nM or less.

Alternatively, the RET inhibitor is for example an antisense RET nucleic acid, a RET specific short-interfering RNA, or a RET-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense RET nucleic acid sequence, an anti-sense RET nucleic acid sequence or both. Optionally, the siRNA is constructed such that a single transcript has both the sense and complementary antisense sequences from the target gene, e.g., a hairpin (shRNA).

ABL kinase inhibitor. In certain embodiments, the ESO Regenerative agent is pan-inhibitor of ABL kinase inhibitor, preferably a BCR-ABL kinase inhibitor. Exemplary pan-inhibitor include imatinib, nilotinib, dasatinib, bosutinib and ponatinib, and is preferably ponatinib.

FLT3 Inhibitors. In certain embodiments, the ESO Regenerative agent is a FLT3 inhibitor. Exemplary FLT3 inhibitors to be used herein are quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

These and further exemplary inhibitors to be used herein are described in more detail below.
Brand Name: Quizartinib

### Structure:

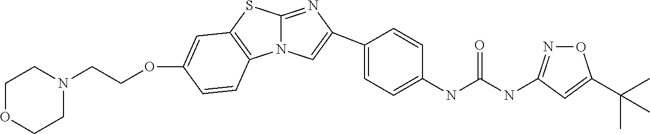 [SMILES: CC(C)(C)c1cc(NC(=O)Nc2ccc(-c3cn4c(n3)sc3cc(OCCN5CCOCC5)ccc34)cc2)no1]

Affinities: FLT3 (1.6 nM), KIT (4.8 nM), PDGFRB (7.7 nM), RET (9.9 nM), PDGFRA (11 nM), CSF1R (12 nM)
Brand Name: Crenolanib

### Structure:

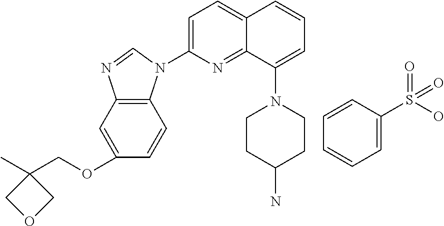

### Affinities: FLT3, PDGFRb

Brand Name: Midostaurin

### Structure:

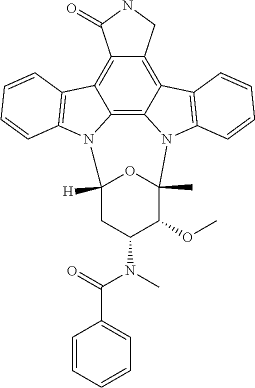 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

Affinities: PKN1 (9.3 nM), TBK1 (9.3 nM), FLT3 (11 nM), JAK3 (12 nM), MLK1 (15 nM), and 30 targets in the range 15-110 nM
Brand Name: Lestaurtinib

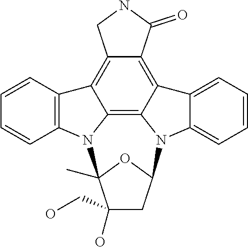 [SMILES: C[C@]12O[C@H](C[C@]1(O)CO)n1c3ccccc3c3c4c(c5c6ccccc6n2c5c31)CNC4=O]

### Affinities: FLT3, TRKA, TRKB, TRKC

Brand Name: 4SC-203

### Structure:

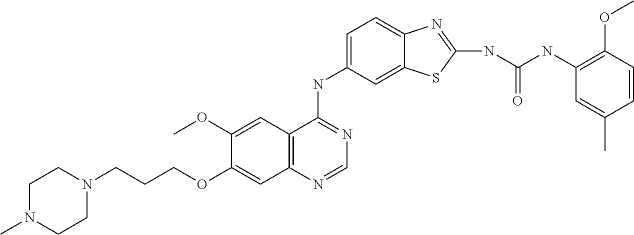

### Affinities: FLT3, VEGFR

### Structure:

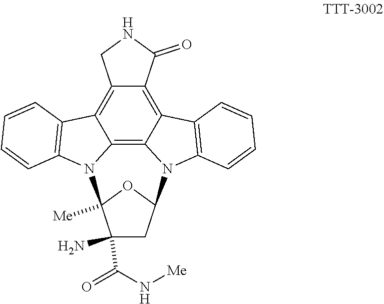 [SMILES: CNC(=O)[C@@]1(N)C[C@H]2O[C@]1(C)n1c3ccccc3c3c4c(c5c6ccccc6n2c5c31)C(=O)NC4]

### Affinities: FLT3 (Wall, Blood (ASH Annual Meeting Abstracts). 2012; 120:866);

LRRK2 (Yao, Human molecular genetics. 2013; 22(2):328-44).

### Clinical Phase: Preclinical

Developer: Tautatis (originator)
Brand Name: Sorafenib

### Code Name: Bay-43-0006

### Structure:

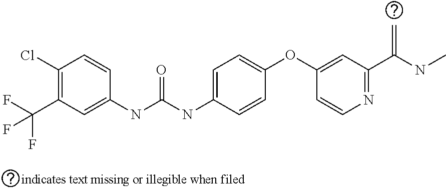 [SMILES: C=C(NC)c1cc(Oc2ccc(NC(=O)Nc3ccc(Cl)c(C(F)(F)F)c3)cc2)ccn1]

IUPAC Name: 4-[4-[3-[4-Chloro-3-(trifluoromethyl)phenyl]ureido]phenoxy]-N-methylpyridine-2-carboxamide
Affinities: DDR1 (1.5 nM), HIPK4 (3 nM), ZAK (6 nM), DDR2 (7 nM), FLT3 (13 nM), and 15 targets in the range 13-130 nM (Zarrinkar, Gunawardane et al. 2009, loc. cit.) Clinical Phase: Launched (renal and hepatocellular carcinoma), Phase I/O (blood cancer) Developer: Bayer
Brand Name: Ponatinib

### Code Name: AP-24534 Structure:

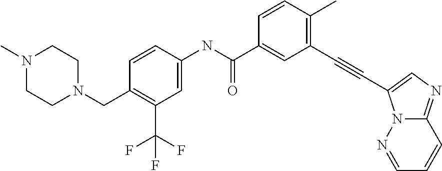 [SMILES: Cc1ccc(C(=O)Nc2ccc(CN3CCN(C)CC3)c(C(F)(F)F)c2)cc1C#Cc1cnc2cccnn12]

IUPAC Name: 3-[2-(Imidazo[1,2-b]pyridazin-3-yl)ethynyl]-4-methyl-N-[4-(4-methylpiperazin-1-ylmethyl)-3-(trifluoromethyl)phenyl]benzamide

### Affinities: BCR-ABL, FLT3, KIT, FGFR1, PDGFRa (Gozgit, Mol Cancer Ther. 2011; 10(6):1028-35).

### Clinical Phase: Phase II (AML)

Developer: Ariad Pharmaceuticals (originator)
Brand Name: Sunitinib

### Code Name: SU-11248

### Structure:

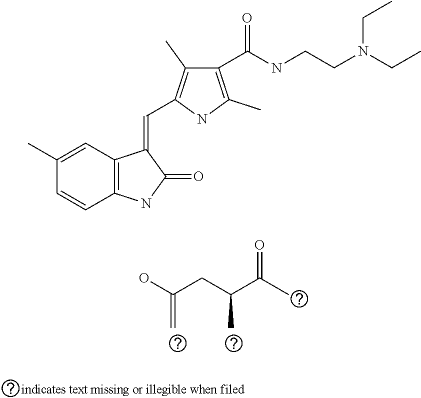

IUPAC Name: (Z)—N-[2-(Diethylamino)ethyl]-5-(5-fluoro-2-oxo-2,3-dihydro-1H-indol-3-ylidenemethyl)-2,4-dimethyl-1H-pyrrole-3-carboxamide 2(S)˜hydroxybutanedioic acid (1:1) N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide L-malate
Affinities: PDGFRB (0.075 nM), KIT (0.37 nM), FLT3 (0.47 nM), PDGFRA (0.79 nM), DRAK1 (1.0 nM), VEGFR2 (1.5 nM), FLT1 (1.8 nM), CSF1R (2.0 nM) (Zarrinkar, Gunawardane et al. 2009, loc. cit.)
Clinical Phase: Launched (renal cell carcinoma, gastrointestinal stromal cancer, neuroendocrine pancreas), phase I (AML)

### Developer: Pfizer (Originator)

Brand Name: Tandutinib

### Code Name: MLN-0518

### Structure:

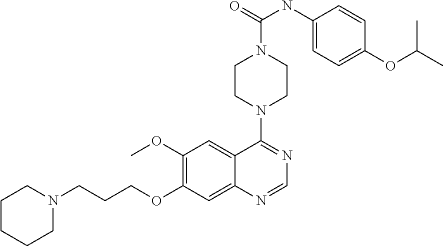 [SMILES: COc1cc2c(N3CCN(C(=O)Nc4ccc(OC(C)C)cc4)CC3)ncnc2cc1OCCCN1CCCCC1]

IUP AC Name: N-(4-Isopropoxyphenyl)-4-[6-methoxy-7-[3-(1-piperidinyl)propoxy]quinazolin-4-yl]piperazine-1-carboxamide
Affinities: PDGFRA (2.4 nM), KIT (2.7 nM), FLT3 (3 nM), PDGFRB (4.5 nM), CSF1R (4.9 nM) (Zarrinkar, Gunawardane et al. 2009, loc. cit.)
Clinical Phase: discontinued

### Developer: Kyowa Hakko Kirin (Originator), Millennium Pharmaceuticals (Originator),

Code Name: FF-10101

### Structure:

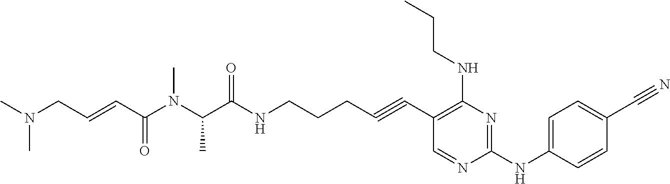 [SMILES: CCCNc1nc(Nc2ccc(C#N)cc2)ncc1C#CCCCNC(=O)[C@H](C)N(C)C(=O)/C=C/CN(C)C]

National Cancer Institute, Takeda (Originator) FLT3 inhibitors to be used in accordance with the present disclosure are not limited to the herein described or further known exemplary inhibitors. Accordingly, also further inhibitors or even yet unknown inhibitors may be used in accordance with the present disclosure. Such inhibitors may be identified by the methods described and provided herein and methods known in the art, like high-throughput screening using biochemical assays for inhibition of FLT3.

Assays for screening potential FLT3 inhibitors and, in particular, for identifying FLT3 inhibitors as defined herein, comprise, for example, in vitro competition binding assays to quantitatively measure interactions between test compounds and recombinantly expressed kinases¹ (Fabian et al; Nat Biotechnol. 2005 23(3):329-36). Hereby, competition with immobilized capture compounds and free test compounds is performed. Test compounds that bind the kinase active site will reduce the amount of kinase captured on solid support, whereas test molecules that do not bind the kinase have no effect on the amount of kinase captured on the solid support. Furthermore, inhibitor selectivity can also be assessed in parallel enzymatic assays for a set of recombinant protein kinases. (Davies et al., Biochem. J. 2000 35(1): 95-105; Bain et al. Biochem. J. 2003 37(1): 199-204). These assays are based on the measurement of the inhibitory effect of a kinase inhibitor and determine the concentration of compound required for 50% inhibition of the protein kinases of interest. Proteomics methods are also an efficient tool to identify cellular targets of kinase inhibitors. Kinases are enriched from cellular lysates by immobilized capture compounds, so the native target spectrum of a kinase inhibitor can be determined.⁴ (Godl et al., Proc Natl Acad Sci USA. 2003 100(26): 5434-9).

Assays for screening of potential inhibitors and, in particular, for identifying inhibitors as defined herein, are, for example, described in the following papers:
FABIAN ET AL., NAT BIOTECHNOL. 2005 23(3):329-36
DAVIES ET AL., BIOCHEM. J. 2000 351: 95-105.
BAIN ET AL., BIOCHEM. J. 2003 371: 199-204.
GODL ET AL., PROC NATL ACAD SCI USA. 2003 100(26): 15434-9.
   The above papers are incorporated herein in their entirety by reference.

### IV. Combination Therapies—Other Agents

In certain embodiments, the IAP Inhibitor agent can be administered conjointly with one or more agents that have other beneficial local activities in esophagus. Illustrative categories and specific examples of active drugs include: (a) antitussives, such as dextromethorphan, dextromethorphan hydrobromide, noscapine, carbetapentane citrate, and chlophedianol hydrochloride; (b) antihistamines, such as chlorpheniramine maleate, phenindamine tartrate, pyrilamine maleate, doxylamine succinate, and phenyltoloxamine citrate; (c) antipyretics and analgesics such as acetaminophen, aspirin and ibuprofen; (d) antacids such as aluminum hydroxide and magnesium hydroxide, (e) anti-infective agents such as antifungals, antivirals, antiseptics and antibiotics, (f) chemotherapeutic agents.

### V. Exemplary Formulations

In certain embodiments, the IAP Inhibitor agents is formulated for topical administration as part of a bioadhesive formulation. Bioadhesive polymers have extensively been employed in transmucosal drug delivery systems and can be readily adapted for use in delivery of the subject IAP Inhibitor agents to the esophagus, particularly the areas of lesions and tumor growth. In general terms, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups responsible for forming hydrogen bonds are the hydroxyl (—OH) and the carboxylic groups (—COOH). When these materials are incorporated into pharmaceutical formulations, drug absorption by mucosal cells may be enhanced and/or the drug may be released at the site for an extended period of time. Merely to illustrate, the bioadhesive can be a hydrophilic polymer, a hydrogel, a co-polymers/interpolymer complex or a thiolated polymer.
Hydrophilic polymers: these are water-soluble polymers that swell when they come in contact with water and eventually undergo complete dissolution. Systems coated with these polymers show high bioadhesiveness to the mucosa in dry state but the bioadhesive nature deteriorates as they start dissolving. As a result, their bioadhesiveness is short-lived. An example is poly (acrylic acid).
Hydrogels: these are three-dimensional polymer networks of hydrophilic polymers which are cross-linked either by chemical or physical bonds. These polymers swell when they come in contact with water. The extent of swelling depends upon the degree of crosslinking. Examples are polycarbophil, carbopol and polyox.
Co-polymers/interpolymer complex: a block copolymer is formed when the reaction is carried out in a stepwise manner, leading to a structure with long sequences or blocks of one monomer alternating with long sequences of the other. There are also graft copolymers, in which entire chains of one kind (e.g., polystyrene) are made to grow out of the sides of chains of another kind (e.g., polybutadiene), resulting in a product that is less brittle and more impact-resistant. Hydrogen bonding is a major driving force for interpolymer interactions.
Thiolated polymers (thiomers): these are hydrophilic macromolecules exhibiting free thiol groups on the polymeric backbone. Based on thiol/disulfide exchange reactions and/or a simple oxidation process disulfide bonds are formed between such polymers and cysteine-rich subdomains of mucus glycoproteins building up the mucus gel layer. So far, the cationic thiomers, chitosan-cysteine, chitosan-thiobutylamidine as well as chitosan-thioglycolic acid, and the anionic thiomers, poly (acylic acid)-cysteine, poly (acrylic acid)-cysteamine, carboxymethylcellulose-cysteine and alginate-cysteine, have been generated. Due to the immobilisation of thiol groups on mucoadhesive basis polymers, their mucoadhesive properties are 2- up to 140-fold improved.

In certain embodiments, the bioadhesive polymer can be selected from poly(acrylic acid), tragacanth, poly(methylvinylether comaleic anhydride), poly(ethylene oxide), methyl-cellulose, sodium alginate, hydroxypropylmethylcellulose, karaya gum, methylethyl cellulose (and cellulose derivatives such as Metolose), soluble starch, gelatin, pectin, poly(vinyl pyrrolidone), poly(ethylene glycol), poly(vinyl alcohol), poly(hydroxyethyl-methacrylate), hydroxypropylcellulose, sodium carboxymethylcellulose or chitosan.

Other suitable bioadhesive polymers are described in U.S. Pat. No. 6,235,313 to Mathiowitz et al., the teachings of which are incorporated herein by reference, and include polyhydroxy acids, such as poly(lactic acid), polystyrene, polyhyaluronic acids, casein, gelatin, glutin, polyanhydrides, polyacrylic acid, alginate, chitosan; polyacrylates, such as poly(methyl methacrylates), poly(ethyl methacrylates), poly butylmethacrylate), poly-(isobutylmethacrylate), poly(hexlmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate); polyacrylamides; poly(fumaric-co-sebacic)acid, poly(bis carboxy phenoxy propane-co-sebacic anhydride), polyorthoesters, and copolymers, blends and mixtures thereof.

In certain embodiments, the bioadhesive is an alginate. Alginic acid and its salts associates with sodium and potassium bicarbonate have shown that, after entering a more acidic environment they form a viscous suspension (or a gel) exerting protecting activity over gastric mucosa. These properties are readily adaptable for topical delivery to the esophagus, particularly the lower esophagus. The scientific and patent literature on its activity is wide. Thus, for example, for delivery to the esophagus: Mandel K. G.; Daggy B. P.; Brodie D. A; Jacoby, H. L., 2000. Review article: Alginate-raft formulations in the treatment of heartburn and acid reflux. Aliment. Pharmacol. Ther. 14 669-690, which is incorporated by reference herein in its entirety; and Bioadhesive esophageal bandages: protection against acid and pepsin injury. Man Tang, Peter Dettmar, Hannah Batchelor—International Journal of Pharmaceutics 292 (2005)-169-177, which is incorporated by reference herein in its entirety.

In certain embodiments, the bioadhesive is a bioadhesive hydrogel. Bioadhesive hydrogels are well known in art and suitable hydrogels that be used for delivery of the IAP Inhibitor agents of the present disclosure are described in a wide range of scientific and patent literature on its activity is wide. An exemplary hydrogel formulation is described in Collaud et al. “Clinical evaluation of bioadhesive hydrogels for topical delivery of hexylaminolevulinate to Barrett's esophagus” J Control Release. 2007 Nov. 20; 123(3):203-10.

Bioadhesive Microparticle formulations. In certain embodiments, the IAP Inhibitor agent (optionally with other active agents) are formulated into adhesive polymeric microspheres have been selected on the basis of the physical and chemical bonds formed as a function of chemical composition and physical characteristics, such as surface area, as described in detail below. These microspheres are characterized by adhesive forces to mucosa of greater than 11 mN/cm² on esophageal tissue. The size of these microspheres can range from between a nanoparticle to a millimeter in diameter. The adhesive force is a function of polymer composition, biological substrate, particle morphology, particle geometry (e.g., diameter) and surface modification.

Suitable polymers that can be used to form bioadhesive microspheres include soluble and insoluble, biodegradable and nonbiodegradable polymers. These can be hydrogels or thermoplastics, homopolymers, copolymers or blends, natural or synthetic. The preferred polymers are synthetic polymers, with controlled synthesis and degradation characteristics. Most preferred polymers are copolymers of fumaric acid and sebacic acid, which have unusually good bioadhesive properties when administered to the gastrointestinal.

In the past, two classes of polymers have appeared to show useful bioadhesive properties: hydrophilic polymers and hydrogels. In the large class of hydrophilic polymers, those containing carboxylic groups (e.g., poly[acrylic acid]) exhibit the best bioadhesive properties. One could infer that polymers with the highest concentrations of carboxylic groups should be the materials of choice for bioadhesion on soft tissues. In other studies, the most promising polymers were sodium alginate, carboxymethylcellulose, hydroxymethylcellulose and methylcellulose. Some of these materials are water-soluble, while others are hydrogels.

Rapidly bioerodible polymers such as poly[lactide-co-glycolide], polyanhydrides, and polyorthoesters, whose carboxylic groups are exposed on the external surface as their smooth surface erodes, are excellent candidates for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers include proteins, such as zein, modified zein, casein, gelatin, gluten, serum albumin, or collagen, and polysaccharides, such as cellulose, dextrans, polyhyaluronic acid, polymers of acrylic and methacrylic esters and alginic acid. These are not preferred due to higher levels of variability in the characteristics of the final products, as well as in degradation following administration. Synthetically modified natural polymers include alkyl celluloses, hydroxyalkyl celluloses, cellulose ethers, cellulose esters, and nitrocelluloses.

Representative synthetic polymers include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

In some instances, the polymeric material could be modified to improve bioadhesion either before or after the fabrication of microspheres. For example, the polymers can be modified by increasing the number of carboxylic groups accessible during biodegradation, or on the polymer surface. The polymers can also be modified by binding amino groups to the polymer. The polymers can also be modified using any of a number of different coupling chemistries that covalently attach ligand molecules with bioadhesive properties to the surface-exposed molecules of the polymeric microspheres.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of bioadhesive ligands to the polymeric microspheres described herein. Any polymer that can be modified through the attachment of lectins can be used as a bioadhesive polymer for purposes of drug delivery or imaging.

Lectins that can be covalently attached to microspheres to render them target specific to the mucin and mucosal cell layer could be used as bioadhesives. Useful lectin ligands include lectins isolated from Abrus precatroius, Agaricus bisporus, Anguilla anguilla, Arachis hypogaea, Pandeiraea simplicifolia, Bauhinia purpurea, Caragan arobrescens, Cicer arietinum, Codiurn fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The attachment of any positively charged ligand, such as polyethyleneimine or polylysine, to any microsphere may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most microspheres with the appropriate chemistry, such as CDI, and be expected to influence the binding of microspheres to the gut. For example, polyclonal antibodies raised against components of mucin or else intact mucin, when covalently coupled to microspheres, would provide for increased bioadhesion. Similarly, antibodies directed against specific cell surface receptors exposed on the lumenal surface of the intestinal tract would increase the residence time of beads, when coupled to microspheres using the appropriate chemistry. The ligand affinity need not be based only on electrostatic charge, but other useful physical parameters such as solubility in mucin or else specific affinity to carbohydrate groups.

The covalent attachment of any of the natural components of mucin in either pure or partially purified form to the microspheres would decrease the surface tension of the bead-gut interface and increase the solubility of the bead in the mucin layer. The list of useful ligands would include but not be limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range would yield chains of 120 to 425 amino acid residues attached to the surface of the microspheres. The polyamino chains would increase bioadhesion by means of chain entanglement in mucin strands as well as by increased carboxylic charge.

As used herein, the term “microspheres” includes microparticles and microcapsules (having a core of a different material than the outer wall), having a diameter in the nanometer range up to 5 mm. The microsphere may consist entirely of bioadhesive polymer or have only an outer coating of bioadhesive polymer.

As characterized in the following examples, microspheres can be fabricated from different polymers using different methods. Polylactic acid blank microspheres were fabricated using three methods: solvent evaporation, as described by E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984); hot-melt microencapsulation, as described by E. Mathiowitz, et al., Reactive Polymers, 6, 275 (1987); and spray drying. Polyanhydrides made of bis-carboxyphenoxypropane and sebacic acid with molar ratio of 20:80 P(CPP-SA) (20:80) (Mw 20,000) were prepared by hot-melt microencapsulation. Poly(fumaric-co-sebacic) (20:80) (Mw 15,000) blank microspheres were prepared by hot-melt microencapsulation. Polystyrene microspheres were prepared by solvent evaporation.

In certain embodiments, the composition includes a bioadhesive matrix in which particles (such as nanoparticles) containing the IAP Inhibitor agents are dispersed. In these embodiments, the bioadhesive matrix promotes contact between the mucosa of the esophagus and the nanoparticles.

In certain embodiments, the drug-containing particle is a matrix, such as a bioerodible, bioadhesive matrix. Suitable bioerodible, bioadhesive polymers include bioerodible hydrogels, such as those described by Sawhney, et al., in Macromolecules, 1993, 26:581-587, the teachings of which are incorporated herein by reference. Representative bioerodible, bioadhesive polymers include, but are not limited to, synthetic polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide), poly(lactide-co-caprolactone), poly(ethylene-co-maleic anhydride), poly(ethylene maleic anhydride-co-L-dopamine), poly(ethylene maleic anhydride-co-phenylalanine), poly(ethylene maleic anhydride-co-tyrosine), poly(butadiene-co-maleic anhydride), poly(butadiene maleic anhydride-co-L-dopamine) (pBMAD), poly(butadiene maleic anhydride-co-phenylalanine), poly(butadiene maleic anhydride-co-tyrosine), poly(fumaric-co-sebacic)anhydride (P(FA:SA)), poly(bis carboxy phenoxy propane-co-sebacic anhydride) (20:80) (poly(CCP:SA)), as well as blends comprising these polymers; and copolymers comprising the monomers of these polymers, and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers, blends and mixtures thereof. In general, these materials degrade either by enzymatic hydrolysis or exposure to water in vivo, by surface or bulk erosion.

Particles having an average particle size of between 10 nm and 10 microns are useful in the compositions described herein. In certain embodiments, the particles are nanoparticles, having a size range from about 10 nm to 1 micron, preferably from about 10 nm to about 0.1 microns. In particularly preferred embodiments, the particles have a size range from about 500 to about 600 nm. The particles can have any shape but are generally spherical in shape.

The compositions described herein contain a monodisperse plurality of nanoparticles. Preferably, the method used to form the nanoparticles produces a monodisperse distribution of nanoparticles; however, methods producing polydisperse nanoparticle distributions can be used. If the method does not produce particles having a monodisperse size distribution, the particles are separated following particle formation to produce a plurality of particles having the desired size range and distribution.

Nanoparticles useful in the compositions described herein can be prepared using any suitable method known in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), coacervation, low temperature microsphere formation, and phase inversion nanoencapsulation (PIN). A brief summary of these methods is presented below.

Spray Drying. Methods for forming microspheres/nanospheres using spray drying techniques are described in U.S. Pat. No. 6,620,617, to Mathiowitz et al. In this method, the polymer is dissolved in an organic solvent such as methylene chloride or in water. A known amount of one or more active agents to be incorporated in the particles is suspended (in the case of an insoluble active agent) or co-dissolved (in the case of a soluble active agent) in the polymer solution. The solution or dispersion is pumped through a micronizing nozzle driven by a flow of compressed gas, and the resulting aerosol is suspended in a heated cyclone of air, allowing the solvent to evaporate from the microdroplets, forming particles. Microspheres/nanospheres ranging between 0.1-10 microns can be obtained using this method.

Interfacial Polymerization. Interfacial polymerization can also be used to encapsulate one or more active agents. Using this method, a monomer and the active agent(s) are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

Hot Melt Microencapsulation. Microspheres can be formed from polymers such as polyesters and polyanhydrides using hot melt microencapsulation methods as described in Mathiowitz et al., Reactive Polymers, 6:275 (1987). In this method, the use of polymers with molecular weights between 3-75,000 daltons is preferred. In this method, the polymer first is melted and then mixed with the solid particles of one or more active agents to be incorporated that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decanting with petroleum ether to give a free-flowing powder.

Phase Separation Microencapsulation. In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

Spontaneous Emulsion Microencapsulation. Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

Solvent Evaporation Microencapsulation. Methods for forming microspheres using solvent evaporation techniques are described in E. Mathiowitz et al., Scanning Microscopy, 4:329 (1990); L. R. Beck et al., Fertil. Steril., 31:545 (1979); L. R. Beck et al., Am J Obstet Gynecol 135(3) (1979); S. Benita et al., Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microspheres/nanospheres. This method is useful for relatively stable polymers like polyesters and polystyrene. However, labile polymers, such as polyanhydrides, may degrade during the fabrication process due to the presence of water. For these polymers, some of the following methods performed in completely anhydrous organic solvents are more useful.

Solvent Removal Microencapsulation. The solvent removal microencapsulation technique is primarily designed for polyanhydrides and is described, for example, in WO 93/21906 to Brown University Research Foundation. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Microspheres that range between 1-300 microns can be obtained by this procedure. Substances which can be incorporated in the microspheres include pharmaceuticals, pesticides, nutrients, imaging agents, and metal compounds.

Coacervation. Encapsulation procedures for various substances using coacervation techniques are known in the art, for example, in GB-B-929 406; GB-B-929 40 1; and U.S. Pat. Nos. 3,266,987, 4,794,000, and 4,460,563. Coacervation involves the separation of a macromolecular solution into two immiscible liquid phases. One phase is a dense coacervate phase, which contains a high concentration of the polymer encapsulant (and optionally one or more active agents), while the second phase contains a low concentration of the polymer. Within the dense coacervate phase, the polymer encapsulant forms nanoscale or microscale droplets. Coacervation may be induced by a temperature change, addition of a non-solvent or addition of a micro-salt (simple coacervation), or by the addition of another polymer thereby forming an interpolymer complex (complex coacervation).

Low Temperature Casting of Microspheres. Methods for very low temperature casting of controlled release microspheres are described in U.S. Pat. No. 5,019,400 to Gombotz et al. In this method, a polymer is dissolved in a solvent optionally with one or more dissolved or dispersed active agents. The mixture is then atomized into a vessel containing a liquid non-solvent at a temperature below the freezing point of the polymer-substance solution which freezes the polymer droplets. As the droplets and non-solvent for the polymer are warmed, the solvent in the droplets thaws and is extracted into the non-solvent, resulting in the hardening of the microspheres.

Phase Inversion Nanoencapsulation (PIN). Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non-solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et al. The method can be used to produce monodisperse populations of nanoparticles and microparticles in a wide range of sizes, including, for example, about 100 nanometers to about 10 microns. Advantageously, an emulsion need not be formed prior to precipitation. The process can be used to form microspheres from thermoplastic polymers.

Sequential Phase Inversion Nanoencapsulation (sPIN). Multi-walled nanoparticles can also be formed by a process referred to herein as “sequential phase inversion nanoencapsulation” (sPIN). This process is described in detail below in Section IV. sPIN is particularly suited for forming monodisperse populations of nanoparticles, avoiding the need for an additional separations step to achieve a monodisperse population of nanoparticles.

Dissolving Tablets or Lozenges. In certain embodiments, the IAP Inhibitor agents is provided in a dissolving tablet. For example, the tablet can contain a therapeutically effective amount of the IAP Inhibitor agent in combination with polyvinylpyrrolidone (PVP: povidone), wherein the tablet is formulated to rapidly dissolve in a specific volume of liquid so as to generate a topical esophageal therapy suitable for delivering the anti-PESC to the luminal surface of the esophagus. For instance, the volume of liquid in which the tablet dissolves can be from 5 to 50 mL, 5 to 25 mL or even 5 to 15 mL. Preferably the liquid is water. The dissolving tablet can also further include an excipient that renders the dissolving tablet palatable, especially at least one excipient that increases viscosity of the topical esophageal therapy. An exemplary viscosity-enhancing excipient is mannitol.

In certain embodiments, the IAP Inhibitor agent is provided in a topical, non-systemic, oral, slow releasing, solid, soft lozenge pharmaceutical composition comprising: (a) about 1% to about 5% by mass of one or more release modifiers comprising polyethylene oxide polymers comprising a molecular weight of about 900,000 to about 8,000,000; (b) about 10% to about 60% by mass of one or more film-forming polymers comprising gelatins; (c) about 5% to about 20% by mass of one or more plasticizers comprising glycerol, sorbitol, or combinations thereof; and (d) less than 1% by mass of one or more IAP Inhibitor agents. Exemplary plasticizers include glycerol, sorbitol, mannitol, maltitol, xylitol, or combinations thereof. The lozenge may also include one or more sweeteners, such as maltitol, xylitol, mannitol, sucralose, aspartame, stevia, or a combination thereof. The lozenge may also include one or more pH modifiers comprising one or more organic acids.

### VI. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Overview

The challenges presented by advanced metastatic cancer have driven efforts to identify and preemptively eliminate precancerous lesions. Here the inventors employ technologies to generate libraries of functionally defined stem cells of endoscopically selected, patient-matched esophageal adenocarcinoma (EAC) and its precursor lesions. Clones from these libraries meet all stem cell criteria, maintain their lesional identity, and display a remarkable and unexpected genome stability at the level of copy number and single nucleotide variation. The high-resolution phylogenetic analysis enabled by these clones defines a successively diminishing mutational threshold for transitions between indolent precursors, a discrete, “advanced” Barrett's, dysplasia, and cancer. Importantly, drug combinations that selectively eliminate Barrett's stem cells derived from multiple patients show similar efficacy against stem cells of “advanced” Barrett's, as well as dysplasia and EAC, suggesting the potential of exploiting indolent precursor lesions to identify common lineage vulnerabilities in more proliferatively aggressive lesions.

### Methods

In vitro stem cell cloning from patient-matched endoscopic biopsies. Under informed consent and IRB-approved protocols at the MD Anderson Cancer Center (IRB 5 IRB00006023; LAB01-543) and the University of Connecticut Health Sciences Center (16-065-03), the inventors obtained therapy-naive samples of esophageal adenocarcinoma (EAC) and its precursor lesions. Cases 1 and 2 were from 1 mm endoscopic biopsies of adjacent lesions deemed to be EAC, Dysplasia, and Barrett's together with normal esophageal mucosa. Tissue from Case 3 was in the form of lung metastases from a primary EAC obtained in pleural effusions. Biopsies or pleural effusion cells were dissociated to single cells as described^{27,28} by digestion in 1 mg/ml collagenase type IV (Gibco, USA) at 37° C. for 30-45 min with agitation. Dissociated cells were passed through a 70 μm Nylon mesh (Falcon, USA) to remove aggregates, washed five times in cold F12 media, and seeded onto a feeder layer of lethally irradiated 3T3-J2 cells in StemECHO media (Multiclonal Therapeutics, Hartford, Conn., USA)²⁸ and grown at 37° C. in a 7.5% CO₂ incubator with media change every 2 days. Colonies appearing in 10 days were digested by TrypLE Express solution (Gibco, USA) for 10-15 min at 37° C. and cell suspensions were passed through 30 μm filters (Miltenyi Biotec, Germany) before passaging onto new feeder lawns. Single cell cloning was performed by fine tip pipetting or by flow sorting into 384-well plates previously seeded with irradiated 3T3-J2 cells.

Stem cell differentiation. Air-liquid interface (ALI) cultures was used to assess stem cell differentiation potential²⁷. Transwell inserts (Corning Incorporated, USA) were coated with 20% Matrigel (BD biosciences, USA) and incubated at 37° C. for 10 min to polymerize. 200,000 irradiated 3T3-J2 cells were seeded to each Transwell insert and incubated at 37° C., 7.5% CO₂ incubator overnight. QuadroMACS Starting Kit (LS) (Miltenyi Biotec, Germany) was used to purify the stem cells by removal of feeder cells. 300,000 stem cells were seeded into each Transwell insert and cultured with stem cell media. At confluency (5 days), the apical media on the inserts was removed through careful pipetting and the cultures were continued in differentiation media (stem cell media without nicotinamide) for an additional 8-14 days prior to harvesting. The differentiation media was changed every one or two days.

Xenografts in immunodeficient mice. All animal experiments were performed in accordance with Institutional Animal Care and Use Committee (IACUC)-approved protocol 16-002 at the University of Houston. Three million stem cells were kept on ice and mixed well with 50% Matrigel (Becton Dickinson, Palo Alto, USA) to a volume of 150 μl and injected subcutaneously in NSG (NODscid IL2ra^{null}) mice (Jackson Laboratories, Bar Harbor, USA). Xenograft size was measured with calipers and the volume was determined by the following formula: tumor volume (mm³)=½×A (mm)×B² (mm²), where ‘A’ represent the largest dimension and ‘B’ indicates the smallest dimension.

Histology and staining. Histology, Hematoxylin and eosin (H&E) staining, Rhodamine staining, Alcian blue staining (VECTOR, USA) and immunofluorescence staining were performed using standard techniques. For immunofluorescence, 4% paraformaldehyde-fixed, paraffin embedded tissue slides were subjected to antigen retrieval in citrate buffer (pH 6.0, Sigma-Aldrich, USA) at 120° C. for 20 min, and a blocking procedure was performed with 5% bovine serum albumin (BSA, Sigma-Aldrich, USA) and 0.05% Triton X-100 (Sigma-Aldrich, USA) in DPBS(−) (Gibco, USA) at room temperature for 1 hour and then immunostained with primary antibodies at 4° C. overnight. The sources of primary antibodies used in this study include: rabbit monoclonal Ki67 (1:500, ab16667, Abcam), rabbit polyclonal Laminin (1:500, ab11575, Abcam), mouse monoclonal Cdh17 (1:300, SC74209, Santa Cruz Biotechnology), goat polyclonal E-Cadherin(1:500, AF648, R&D Systems). All images were captured by using the Inverted Eclipse Ti-Series (Nikon, Japan) microscope with Lumencor SOLA light engine and Andor Technology Clara Interline CCD camera and NIS-Elements Advanced Research v.4.13 software (Nikon, Japan) or LSM 780 confocal microscope (Carl Zeiss, Germany) with LSM software. Bright field cell culture images were obtained on an Eclipse TS100 microscope (Nikon, Japan) with Digital Sight DSFi1camera (Nikon, Japan) and NIS-Elements F3.0 software (Nikon, Japan).

DNA content analysis. Stem cells were harvested and washed twice with cold phosphate-buffered saline (PBS). After fixation in 70% cold ethanol at −20° C. for at least 1.5 h, the samples were stained using Propidium Iodide Flow Cytometry Kit (ab139418) and then analyzed by SH800 FACS Cell Sorter (Sony, Japan).

Whole exome sequencing. For exome capture and high-throughput sequencing, about 1 ug of genomic DNA was extracted using QIAGEN kits. The genomic DNA was sheared, end-repaired, A-tailed, adaptor-ligated, and Exome captured using Agilent SureSelect Human All Exon V6 Kit (Agilent Technologies, CA, USA) following the manufacturer's recommended protocols. In short, fragmentation was conducted by hydrodynamic shearing system (Covaris, Massachusetts, USA) to generate 180-280 bp fragments. Remaining overhangs were converted into blunt ends via exonuclease/polymerase activities. After adenylation of 3′ ends of DNA fragments, adapters were ligated. Fragments with ligated adapters on both ends were selectively enriched in a PCR reaction. Captured libraries were enriched in a PCR reaction to add indexes to prepare for hybridization. Products were purified using AMPure XP system (Beckman Coulter, Beverly, USA) and quantified with the Agilent high-sensitivity DNA assay on the Agilent Bioanalyzer 2100 System. The multiplexed libraries were sequenced on Illumina HiSeq X platform (150 bp paired-end reads, Illumina, California, USA). The clusters that do not pass the Chastity filter were removed from downstream analysis. At least 20 million paired reads were generated for each sample.

Low-pass whole genome sequencing. Sequencing libraries were prepared by TruSeq Nano DNA HT Sample Prep Kit (Illumina, California, USA) following the manufacturer's protocol. First, 1000 ng of genomic DNA was fragmented by sonication to 350 bp. Then fragments were end-repaired, A-tailed and adaptor-ligated, followed by further PCR reactions. After purification using the AMPure XP system (Beckman Coulter, Beverly, USA), the library was size-selected using Agilent 2100 Bioanalyzer and quantified by real-time PCR. The clustering of the index-coded samples was performed on a cBot Cluster Generation System using Hiseq PE Cluster Kit (Illumina, California, USA) according to the manufacturer's standard protocol. Next, the libraries were sequenced on Illumina Hiseq X platform (Illumina, California, USA) in 150 bp paired-end model. At least 20 million paired-end reads were generated for each sample.

SNV/Indel/CNV and ploidy calling. Data preprocessing. The raw sequencing reads were quality controlled by removing the adapters' bases and the low-quality bases (Phred-value<10) from the read ends and by discarding the reads with >10% ambiguous bases inside using Trimmomatic⁵¹ version 0.36. Murine sequences were filtered using xenome⁵² version 1.0.1 with default parameters. The remaining reads were aligned to human reference genome (UCSC hg19) using BWA⁵³ version 0.7.15-r11403 under the mem model requiring map quality >=40 (Phred-value). PCR duplicates were removed using Picard tool version 2.15.0 (broadinstitute.github.io/picard/). The GATK^{54,55} version 3.8.04 was used to realign the reads near indels (Mills_and_1000G_gold_standard indels bundled within GATK pipeline) and to recalibrate the base qualities with default settings following the best practice protocol⁵⁵.

SNVs/Indels calling. SNVs and Indels were called by software Manta⁵⁶ version 1.3.25 and Strelka^{57,58} version 2.9.26 with default parameter values in somatic calling model. Only the SNVs/Indels that passed the default filter of Manta and Strelka in derived vcf files were used in downstream analyses. The inventors also applied harder filters to the variants that require only two genotypes presence, variant quality (Phred-value)>30, total read depth >15, alternative allele depth >5, and alternative allele proportion >5%. Also, they required that the corresponding matched normal sample were homozygous wild type at the mutation sites. Somatic mutations were further filtered to remove possible germline mutations based on a panel of 27 normal samples. Somatic mutations with allele frequencies less than 0.01 in 1000 Genome database or gnomAD database were discarded as well. SNVs and Indels were annotated with ANNOVAR web version.

CNV calling. The GATK⁵⁹ somatic copy number variants calling pipeline version 4.0.4.0 (gatkforums.broadinstitute.org/gatk/discussion/9143/) was used to call the CNVs. The inventors used 17 normal female samples sequenced on the same platform to build the CNV panel of normals (PoN) with extra parameter “—minimum-interval-median-percentile 10.0”. The contigs shorter then 46709983 bp were excluded for further analysis. The 1000G phase1 high-quality SNPs (1000G_phase1.snps.high_confidence bundled within GATK pipeline) was used to collect allelic counts information. In the segmentation step, the inventors used patient-matched normal samples and applied parameters “—number-of-smoothing-iterations-per-fit 1—minimum-total-allele-count 15—window-size 7500”. The other steps used the default settings. Segments with less than 15 SNVs were excluded. After getting the segmented confidence interval of copy ratio and allele fraction information, the absolute allelic copy number was inferred and curated manually by considering the consistence of copy ratio and allele fractions and the unique features in different copy number (CN) patterns (e.g., CN1's allele fraction=0 or 1, CN3's allele fraction=0.33 or 0.66). Briefly, after generating segmented copy-ratio (sCR) and allele-fraction (sAF) results from GATK pipeline, the inventors first determined the absolute copy number for CN0 (copy number=0), CN1, and CN2 regions by genome-wide analysis of the raw sequencing reads at germline heterozygous sites in the respective Barrett's, Dysplasia, and EAC clones based on the expectation that CN0 regions have no sequencing reads regardless of amplification of surrounding regions, CN1 regions are homozygous, and some of the CN2 regions show heterozygosity. Then, based on the evenly distributed copy-ratio peaks and clearly separated confidence intervals of each peak, as well as its consistency with sAF patterns, the inventors assigned absolute copy number integers to CN3, CN4, CN5, etc. For instance, with Dysplasia clone D1-5, considering even spacing of sCR patterns (close to 0.35), they assigned CN0 to position R5 lacking reads, CN1 to R8 due to homozygosity (sCR=0.35, sAF=0.00/0.99), CN2 to R6 on account of heterozygosity (sCR=0.71, sAF=0.48/0.51), CN3 to R2 (sCR=1.05, sAF=0.32/0.67), CN4 to R1 (sCR=1.34, sAF=0.24/0.75), CN5 to R9 (sCR=1.71, aAF=0.40/0.59. The CNV result was further confirmed by ABSOLUTE³⁷ algorithm with default parameters. In selecting the best model of ABSOLUTE output, the inventors required that each copy number peak should be under an integral number, the bottom peak for copy number should be close to zero, and the ploidy value should be very close (>0.95) to 1 because the data were obtained from single cell-derived clones. The top model meeting these criteria was considered as the best model.

Ploidy calling. After getting the copy number profiles, the inventors multiplied each segment's absolute copy number to its proportion of the genome in length and added up the derived products as the ploidy number.

Phylogenetic tree construction and ordering of somatic mutations. Ternary genotypes of filter-passed somatic SNVs identified from all WES data by Strelka were used in phylogenetic tree construction. The genotypes of normal sample (e.g., matched blood or fibroblast) were added as an outgroup. The trees were built by SiFit⁶⁰ that employs a heuristic search algorithm to infer the Maximum Likelihood (ML) phylogenetic tree under a finite-site model of evolution. The number of iterations was set to 10000. The “InferAncestralStates” program of SiFit was used for inferring the order of somatic mutations on the branches of the phylogeny based on the false negative rate, deletion rate, and LOH rate reported by SiFit during learning the tree in tree building step.

Clonality analysis. To ensure that each pedigree was single cell-derived, the inventors analyzed the distribution of variant allele fractions (VAFs) of the identified somatic mutations in each sample. For monoclonal pedigrees, diploidy (2n) pedigrees' VAFs should distribute around 50% and triploidy (3n) pedigree will have VAFs around 0.33 and 0.66. For polyclonal pedigrees, most of somatic mutations should have VAFs less than 0.5. Polyclonal pedigrees were excluded from further analysis.

Expression analysis. Total RNAs were extracted from immature stem cell colonies for microarray analysis. RNAs were amplified using WT Pico RNA Amplification System V2 and Encore Biotin Module (NuGEN Technologies, CA, USA). All samples were prepared according to manufacturer's instructions and hybridized onto GeneChip Human Exon 1.0 ST array (Affymetrix, CA, USA). GeneChip operating software was used to process all the Cel files and Affymetrix Expression Console software was used for quality control analysis of microarray data. The gene expression analysis was performed using Partek Genomics Suite 6.6 (Partek Incorporated, USA). All the probe intensity values were normalized and log 2-transformed. To identify the differentially expressed genes, 1-way ANOVA was performed (cutoff value: log₂ fold-change >1.5 and p<0.05). All the comparisons were performed as a pairwise manner and gene sets from each comparison were overlapped and selected the unique gene signature for each sample. Unsupervised clustering and heatmap generation were performed with sorted datasets by Euclidean distance based on average linkage clustering, and Principal Component Analysis (PCA) map was made using all probe sets. Pathway enrichment analysis was performed using Enrichr⁶¹.

### Results

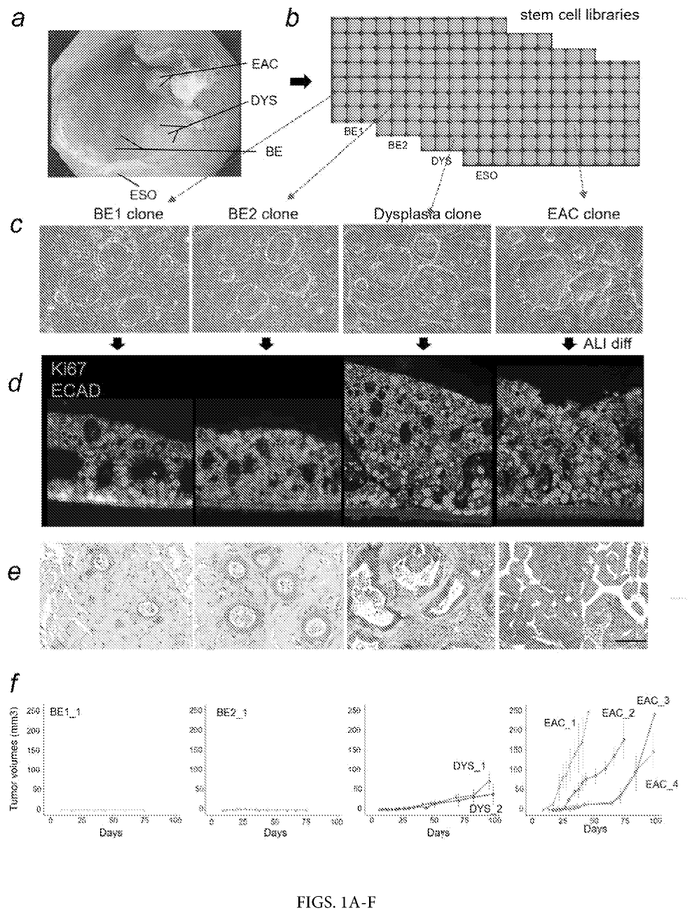

Clonogenic cells from patient-matched lesions. A series of 1 mm endoscopic biopsies from adjacent regions of Barrett's, dysplasia, and esophageal adenocarcinoma was obtained from therapy-naive patients suspected of early esophageal adenocarcinoma (FIG. 1a). Each biopsy was dissociated to yield 100,000 to 500,000 epithelial cells and plated onto lawns of irradiated 3T3-J2 fibroblasts to generate libraries of 100 to 500 epithelial colonies after 10 days of growth^{15,29,30}. The plating efficiency of the epithelial cells from these biopsies indicated that 1:1,000 to 1:5,000 of these cells can form colonies in the culture system, a number similar that of clonogenic cells from normal intestinal mucosa²⁹. Single cell-derived clones from these libraries were obtained by flow-sorting to 384-well plates (FIG. 1b) and could be propagated as discrete lines for at least one-year (FIG. 1c) with a clonogenicity between 25-50 percent. To further characterize these clones, the inventors triggered their differentiation in air-liquid interface (ALI) cultures²⁷ known to produce three-dimensional (3D) epithelia (FIG. 1d). Clones from Barrett's biopsies gave rise to intestinal metaplasia marked by either high or moderate cell polarity, whereas the dysplasia and EAC clones differentiated to densely cellularized epithelia having higher levels of the of the proliferation marker Ki67 and a general loss of cell polarity. Transplantation of these same clones into highly immunodeficient (NODscid IL2rg^{null}; NSG) mice²⁹ yielded nodules with histological characteristics of Barrett's esophagus, dysplasia, and esophageal adenocarcinoma (FIG. 1e). Despite the absence of polarity in ALI-generated epithelia from both dysplasia and EAC stem cells, only EAC clones formed aggressive tumors in these mice, whereas dysplasia clones formed more indolent, cyst-like nodules (FIGS. 1e-f).

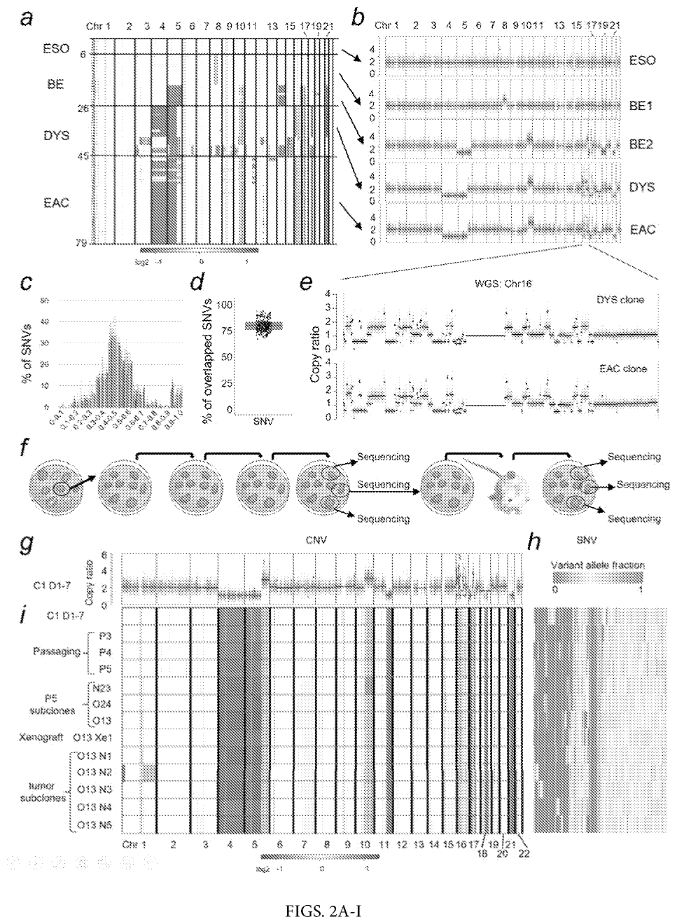

Interclonal heterogeneity and clonal genomic stability. To assess clonal heterogeneity within and across these lesion-specific stem cell libraries, the inventors selected 76 single cell-derived clones from Case 1 (6 esophageal, 20 Barrett's, 19 dysplasia, and 32 EAC) for expansion and low-pass, whole-genome sequencing (lpWGS; ave. 1.6× coverage; FIG. 2a). Inspection of copy number ratio profiles showed that the esophageal clones lacked CNV, whereas the Barrett's, dysplasia and EAC clones all showed multiple and often similar CNV events. In particular, a subset of Barrett's clones, deemed hereafter as “advanced Barrett's” or “BE2” Case 1 showed CNV events impacting Chr. 5, 10, 17, and 21 that were also present in some of the dysplastic clones and all EAC clones (FIG. 2a). To examine the potential relationships between these clones in more detail, the inventors sampled 35 of these 76 clones for whole exome sequencing (WES, ave. 120× coverage; FIG. 2b). Single nucleotide variation (SNV) analyses of these 35 clones showed allele frequencies that hovered around 0.5, consistent with the derivation of these clones from single cells (FIG. 2c). Importantly, the inventors found that most synonymous and nonsynonymous mutations harbored by a clone from a particular biopsy of Barrett's, dysplasia, and EAC were shared among the independently derived clones from the same biopsy, supporting the notion that these mutations preexisted in the cells of the biopsy (FIG. 2d). A similar conclusion applies to the major CNV events seen in these clones that are common to clones from and often across distinct lesions (op. cit. FIGS. 2a-b).

While these clones seemed to accurately reflect the mutational profiles of the neoplastic cells in the patient biopsies, it was less clear whether the known genomic instability of cancers³²⁻³⁴ would, over extended growth, degrade the proxy value of these clones. In this regard, the inventors noted that some of the dysplasia clones and all EAC clones displayed a chromothripsis event of chromosome 16 marked by complex rearrangements and translocations (e.g., FIG. 2b)³⁵⁻³⁶. Assuming that this chromothripsis event might be a sentinel for genomic instability, the inventors examined whole genome sequencing (WGS, 40× coverage) profiles of one dysplastic clone (A1S-12) and one EAC clone (D1-1C) that likely diverged several years apart in the patient^{37,38}. Remarkably, the structure of chromosome 16 assembled from WGS of these two clones was largely indistinguishable (FIG. 2e). This finding led us to question the overall genomic stability of Case 1 clones across extensive propagation in vitro and as xenografts in mice. To address this issue, the inventors asked how the genomic profiles of individual clones (e.g., EAC clone C1-D1-6) varied over multiple cell divisions during serial passaging in vitro and after 6 weeks as xenografts in immunodeficient mice (FIGS. 2f-g). At discrete passages of in vitro cultivation, and after tumor formation in xenografts, the inventors re-cloned cells through the generation of libraries of clonogenic cells and by flow-sorting to single cells (FIG. 2f). DNA was collected from the derived subclones and subjected to WES (142× coverage). Surprisingly, both the dysplasia and EAC clones showed little in the way of arm-level or whole chromosome loss or gains either in vitro or during growth as xenografts in vivo (FIG. 2i). In addition, these clones showed minimal changes at the single nucleotide level within exons following long-term passaging in vitro or as xenografts in vivo, with a complete conservation of the starting 82 nonsynonymous SNPs and gain of an average of 7 SNPs in 50 days in culture and 6 SNPs during 41 days of tumor growth in mice. These data suggest that the dysplasia and EAC clones showed a genomic stability like that of normal gastrointestinal stem cells²⁷ and that these clones, in aggregate, reflect the genomics of the disease.

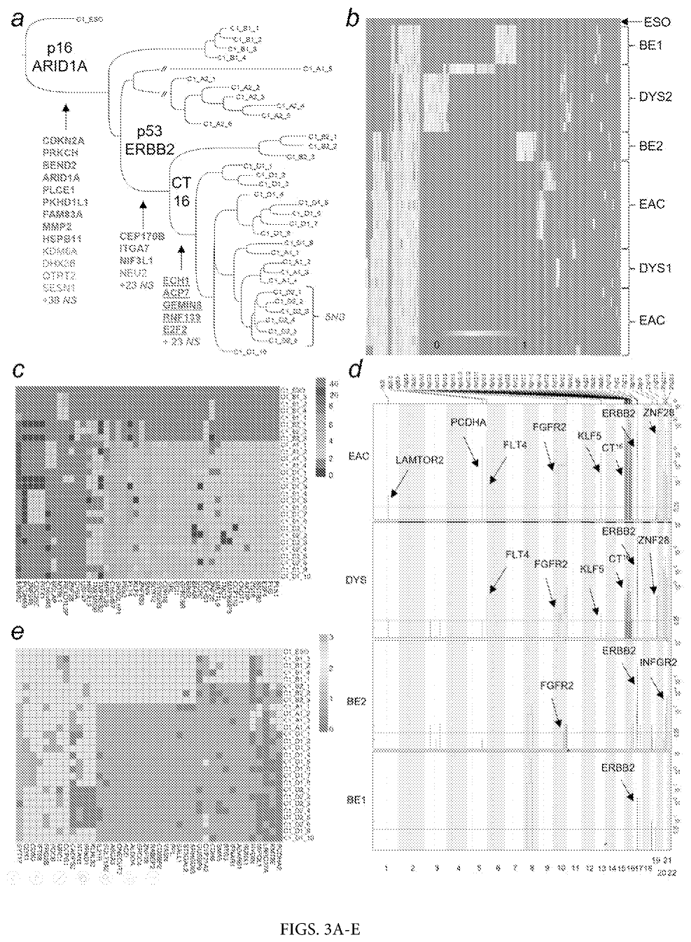

Clonal phylogenetics to cancer. To assess the evolutionary relationships between the Barrett's, Dysplasia, and EAC clones, the inventors performed a phylogenetic analysis across the 35 clones with WES data of Case 1 based on 679 somatic SNVs (allele frequency >0.2) from the WES data of the 35 clones (FIGS. 3a-b), most of which, as expected, were heterozygous mutations with variant allele fractions (VAF) around 0.5 (FIG. 3b). The resulting 6 clades in the phylogenetic tree suggested a common ancestor evolving into the “in-line” clades (BE1, BE2, DYS1, and EAC2) that ultimately led to the tumor in this patient and one additional clade (DYS2) that did not contribute to presenting tumor. In addition to p16 and ARID1A mutations associated with Barrett's esophagus⁹, the BE1 clones harbored 49 somatically-derived, code-altering mutations (CAMs; nonsynonymous SNVs, stop-gain, and indels) that were transmitted to the more advanced, “BE2” clones, as well as many others acquired by BE1 clones after the generation of BE2 clones. The inventors also noted an amplification of the ERBB2 locus in all BE2, DYS, and EAC clones (FIGS. 3c-d), and one with the same breakpoints in one of the four BE1 clones (B1-2: 6× ERBB2 amplification). The BE2 clones showed a decidedly more ominous mutational profile. Among changes in BE2 clones that were ultimately transmitted in line to dysplasia clones were p53 mutations (stop-gain/deletion), a further fold amplification of the ERRB2 locus to 14 copies, 27 additional CAMs, and 15 additional CNV events affecting 592 genes. In addition to the 49 CAMs from BE1 and the 27 CAMs from BE2, the in-line dysplasia (DYS1) clones showed the development of a chromothripsis event impacting chromosome 16 (Chr16), acquired an additional 28 CAMs, as well as 8 new CNV events impacting 214 genes, all of which were transmitted to EAC clones. Finally, the in-line transition from dysplasia to EAC was accompanied by only 5 additional nonsynonymous mutations, a further amplification of the ERRB2 locus to 35-40 copies, and only one new CNV event affecting 53 genes. Importantly, all clones shared 42 of the 49 CAMs found in the BE1 ancestor, underscoring the link between the precursor clones and the 16 EAC clones analyzed. While the clones of the DYS2 clade did not give rise to the tumor in this patient, their mutational profile involving p53, ARID1A, ARIDB, ERBB2, as well as a host of other genes underscores their potential for progression.

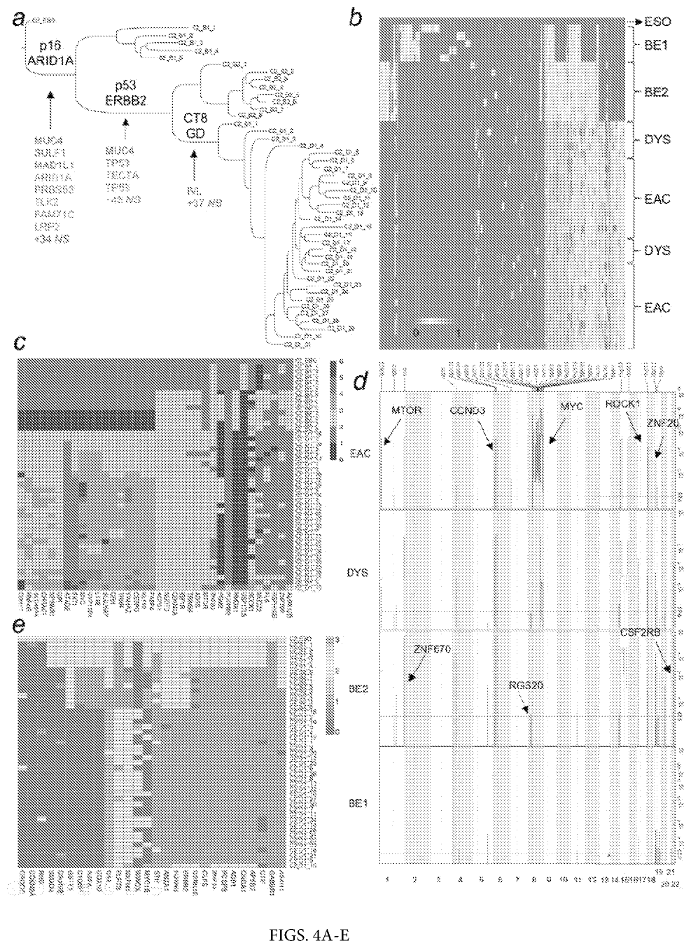

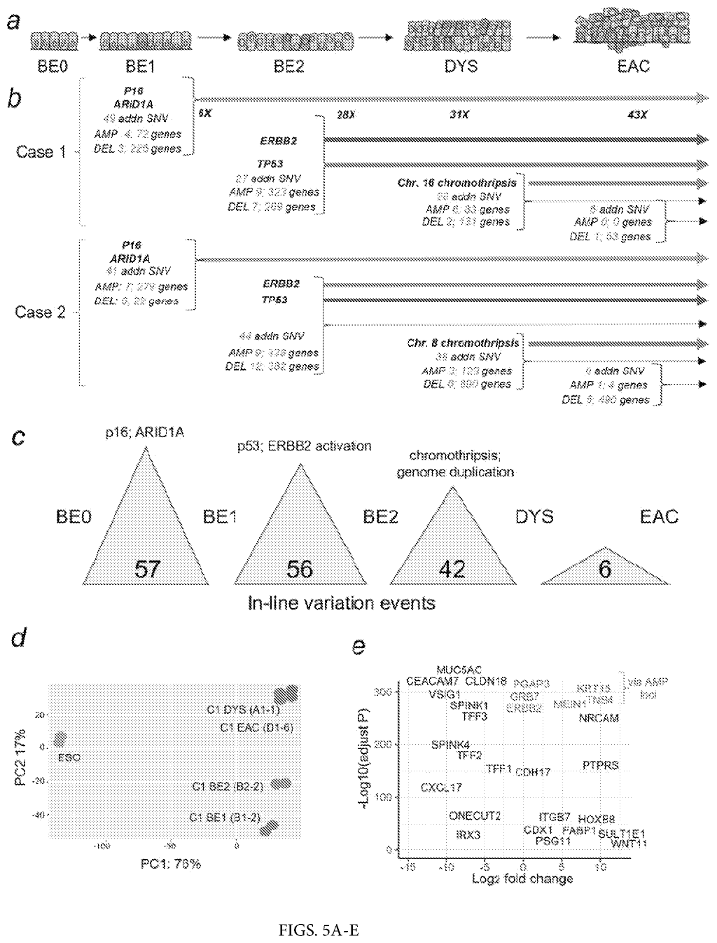

From a second case of esophageal adenocarcinoma, the inventors determined the phylogenetic relationships between 45 clones sampled from libraries based on 463 somatic SNVs (FIGS. 4a-b). This analysis showed four in-line clades that correspond to the BE1, BE2, DYS, and EAC clades seen in Case 1, and are linked by the absolute presence of 35 of the 42 CAMs identified in BE1 of Case 2. As in Case 1, the BE1 clones showed a biallelic loss of p16, a non-synonymous mutation in ARID1A, and 41 CAMs, all of which were passed on to BE2. Like Case 1, the transition to advanced Barrett's (BE2) was accompanied by mutations in p53 (stop-gain/deletion) and ERBB2 activation, the latter via a nonsynonymous (G776V)³⁷ mutation. In addition, BE2 in Case 2 acquired an additional 44 CAMs, as well as 21 interstitial CNV events impacting 720 genes (FIGS. 4a-d, FIG. 5b), all of which were passed on to DYS clones. The transition to dysplasia in Case 2 was, as with Case 1, accompanied by the development chromothripsis event (Chr. 8), as well as the acquisition of 38 CAMs and 9 CNV events impacting 1013 genes. Unlike Case 1, dysplasia in Case 2 was marked by a genome duplication event, a phenomenon common to more than 50% of EACs^{40,42}. While these mutations in dysplasia were transmitted EAC, the transition to EAC was remarkable for its lack of addition CAMs and the acquisition of only 6 CNV events affecting 494 genes.

BE2 and thresholds in oncogenesis. While detailed clonogenic analyses were limited to two cases of EAC, the parallels seen in the transitions from BE1, BE2, DYS, and EAC clones suggest patterns that likely have clinical correlates (FIGS. 5a-b). For instance, Barrett's esophagus is a common clinical finding in an estimated 1-3% of individuals in Western countries and yet the risk of progression to EAC of uncomplicated Barrett's is low (ca. 0.1% per year)^{1,42.} In contrast, individuals with dysplastic Barrett's show a very high risk of progression to EAC (ca. 10%/yr) and the finding triggers immediate intervention in the form of radiofrequency ablation (RFA) or endoscopic mucosal resection (EMR)^{1,35,38}. The clinical risk assessment of Barrett's esophagus is consistent with the behavior and mutational profile of the BE1 clones (non-tumorigenic; wild type p53, absence of amplified protooncogenes). As well, the high-risk associated with dysplastic Barrett's is consistent with the mutational profiles of the DYS clones (mutant p53, multiple protooncogene amplifications, chromothripsis events, along with other changes) and the very minimal changes that distinguish DYS from EAC clones.

Aside from low-risk Barrett's esophagus and high-risk dysplasia, there is much clinical interest in the presence of histological intermediates known as “low-grade dysplasia (LGD)” and “indeterminant for dysplasia” as a harbinger for progression. While the inter-observer agreement for LGD can be low, there is general agreement that LGD has an enhanced risk (0.4-13.4%/year) for progression to dysplasia and EAC^{43,44}. The BE2 clones identified from both EAC cases examined here display a partial loss of polarity upon differentiation in 3-D cultures consistent with LGD, and show a mutational profile (p53 mutations, ERRB2 amplifications or mutational activation, multiple CAMs and CNV events) that would conceivably enhance its risk for further progression over BE1. To identify BE2 biomarkers that could aid in the detection of LGD, the inventors compared whole genome expression profiles of 3-D epithelia formed by BE1 (BE1-5) and BE2 (BE2-8) clones. A volcano plot of these data indicates that BE1 epithelia express known markers of Barrett's esophagus (e.g., TFF1, TFF2, and TFF3, SPINK1 and SPINK4, and CLDN18), whereas BE2 epithelia express an array of genes, exclusive of those in amplified loci, including NRCAM, CEACAM6, CDH17, PTPRS, and FABP1, among many others. The inventors anticipate that a panel of such biomarkers could aid in the detection of BE2 clones in a field of BE1 clones to stratify risk in patients with Barrett's esophagus.

Small molecule screens against precursor stem cells. Given that Barrett's esophagus is an essential precursor for EAC, the inventors adapted BE1 stem cells to high-throughput screening platforms to identify proof-of-concept leads for preemptive therapies. Parallel screens of BE1 and normal esophageal stem cells in 384-well plates against small molecule collections yielded off-diagonal a set of off-diagonal nominal hits of which many showed differential lethality against BE1 stem cells. However, the best of these molecules showed upon dose-response assays to have an 20-fold IC₅₀ advantage over normal esophageal stem cells. In the process of screening, the inventors noted several compounds that enhanced the growth of the normal esophageal stem cells while marginally inhibiting the growth of the target BE1 stem cells. Across screens of Barrett's esophagus stem cells from eight cases, the inventors identified the tyrosine kinase inhibitor ponatinib⁴⁵ as the best of these esophageal stem cell “promoters”. The inventors rescreened the BE1 and normal esophageal stem cells in the presence of ponatinib to yield a new set of hits that effectively inhibited BE1 stem cells while sparing the esophageal stem cells. One of these, SM-164, is an inhibitor of XIAP, one of a set of 8 IAP proteins known to regulate caspase-mediated cell death⁴⁷. In combination with ponatinib, SM-164 effectively eliminates BE1 stem cells with an IC₅₀ of less than 1 nM with minimal impact on normal esophageal stem cells. In co-cultures of BE1 and normal esophageal stem cells that potentially mimic the interactions between these cells in the distal esophagus, this drug combination selectively eliminates the BE1 cells while promotes the expansion of the normal esophageal stem cells.

Given the efficacy of the SM-164/ponatinib combination against BE1 stem cells, the inventors asked if it would have any effect on stem cells of more advanced BE2, DYS, and EAC lesions. Remarkably, the combination showed similar efficacy against the entire lineage of BE1 to EAC even though these compounds were identified for their effect on BE1.

### Discussion

The present work applied technology for single cell cloning of normal mucosal stem cells to multiple, patient-matched lesions implicated in the oncogenesis of esophageal adenocarcinoma. The salient features of the cells cloned from these lesions, including high clonogenicity, unlimited proliferative capacity, and absolute fate commitment to the respective BE1, BE2, DYS, and EAC lesions both in vitro and in vivo, generalizes the cancer stem cell concept to all lesions in oncogenesis^{15,46,47}. The clonal analysis afforded by these cells demonstrates that the vast majority of single nucleotide and copy number variation events present in these clones preexisted in the patient's lesion and were not a consequence of the adaptation of these cells to culture. Moreover, tracking individual DYS and EAC clones through extensive propagation accompanying serial passaging in vitro and clonal tumors in vivo reveal their immense and unexpected genomic stability at both CNV and SNV levels. These features enabled a high-resolution assembly of the phylogenetic relationships between these patient-matched precursor lesions and the presenting tumor which likely evolved over years and even decades. In the two EAC cases assessed in detail, this analysis revealed a discrete clade of stem cells that evolved from BE1 and gave rise to DYS clones. This intermediate, termed “BE2” was distinguished from the BE1 clones by the loss of p53 and the gain of ERBB2 activity, in addition to a host of other single nucleotide and copy number variation events, and likely corresponds to the clinical entity of “low-grade dysplasia” associated with enhanced risk for progression to high-grade dysplasia and EAC^{1,43,44}. The inventors' comparison of the gene expression profiles of these BE1 and BE2 clones has identified a common panel of genes across these two patients whose expression could assist in the identification of patients with Barrett's esophagus who are at risk for progression to dysplasia and EAC. An examination of the in-line mutational profiles across the BE1, BE2, DYS, and EAC clades revealed major changes from BE1 to BE2 and from BE2 to DYS, but very minimal changes from DYS to EAC, the latter amounting to a small number of new code-altering mutations and few or no CNV events. Overall, the magnitude and specificity of the mutational profiles in each of the transitions argues that once the BE2 stage is achieved, the subsequent transitions to DYS and to EAC seem progressively more likely. These findings support the early screening for Barrett's and especially BE2, as well as the development of therapeutics that target the discrete stem cell populations of these lesions. Lastly, the stem cells cloned in this work are likely essential for the future regenerative growth of the lesions from which they were derived, and therefore represent fitting targets for both preemptive and post facto therapeutics.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.

### References for Results and Discussion

1. Sharma P, et al., Gastroenterology 149, 1599-1606 (2015).
2. Shain Ah, et al., N. Engl. J. Med. 373, 1926-1936 (2015).
3. Fischer C, Wood L. J Pathol. 246, 395-404 (2018).
4. Vogelstein B, et al., N Engl J Med. 319, 525-532 (1988).
5. Yokoyama A, et al., Nature 565, 312-317 (2019).
6. Fearon E, And Vogelstein B. Cell 61, 759-767 (1990).
7. Correa P. Cancer Res. 52, 6735-6740 (1992).
8. Greaves M, And Maley C. Nature 481, 306-313 (2012).
9. Reid B, et al., Gastroenterology 149, 1142-1152 (2015).
10. Vogelstein B, et al., N Engl J Med. 373, 1895-1898 (2015).
11. Cancer Genome Atlas Research Network. Comprehensive Molecular Characterization Of Gastric Adenocarcinoma. Nature 513, 202-209 (2014).
12. Tan P, and Yeoh K. Gastroenterology 149, 1153-1162 (2015).
13. Barrett, N. Gastroenterologia 86, 183-186 (1956).
14. Naef A, et al., J. Thorac. Cardiovasc. Surg. 70, 826-835 (1975).
15. Yamamoto Y, et al., Nat Commun. 7:10380 (2016).
16. Maley C, et al., Cancer Epidemiol. Biomark. Prev., 1375-1384 (2004).
17. Maley C, et al., Nat. Genet. 38, 468-473 (2006).
18. Izzo J, et al., Semin Oncol. 34(2 Suppl 1):S2-6 (2007).
19. Galipeau P, et al., J Natl Cancer Inst. 91(24), 2087-2095 (1999).
20. Dulak, A, et al., Nat. Genet. 45, 478-486 (2013).
21. Frankell, A, et al., Nat. Genet. 51(3):506-516 (2019).
22. Stachler M, et al., Nat Genet. 47, 1047-1055 (2015).
23. Ross-Innes C, et al., Nat Genet. 47, 1038-1046 (2015).
24. Gerlinger M, et al., N. Engl. J. Med. 366, 883-892 (2012).
25. Martincorena I, et al., Science 348, 880-886 (2015).
26. Navin N, et al., Nature 472, 90-94 (2011).
27. Zhang J, et al., Science 346, 256-259 (2014).
28. Mcgranahan N, and Swanton C. Cell 168, 613-628 (2017).
29. Wang X, et al., Nature 522, 173-178 (2015).
30. Duleba M, et al., Gastroenterology 156, 20-23 (2019).
31. Pearson T, et al., Curr Top Microbiol Immunol. 324, 25-51 (2008).
32. Ganem N, et al., Curr Opin Genet Dev. 17, 157-162 (2007).
33. Ben-David U, et al., Nat Rev Cancer 19, 97-109 (2019).
34. Bakhoum S, and Cantley L. Cell 174, 1347-1360 (2018).
35. Stephens P, et al., Cell 144, 27-40 (2011).
36. Leibowitz M, et al., Annu Rev Genet. 49, 183-211 (2015).
37. Sharma P, et al., Gastroenterology. 158, 760-769 (2020).
38. Rastogi A, et al., Gastrointest Endosc. 67, 394-398 (2008).
39. Nagano M, et al., Clin Cancer Res. 24, 5112-5122 (2018).
40. Carter S, et al., Nat. Biotechnol. 30, 413-421 (2012).
41. Bielski C, et al., Nat Genet. 50, 1189-1195 (2018).
42. Dam A, et al., Ann Transl Med. 8, 1107 (2020).
43. Duits L, et al., Gut 64, 700-706 (2015).
44. Kestens C, et al., Clin Gastroenterol Hepatol. 14, 956-962.E1 (2016).
45. Tan F, et al., Onco Targets Ther. 12, 635-645 (2019).
46. Lu J, et al., Cancer Res. 68, 9384-9393 (2008).
47. Silke and Meier, 2013.
48. Wang J, and Dick J. Trends Cell Biol. 15, 494-501 (2005).
49. Reya T, et al., Nature 414, 105-111 (2001).
50. ≈
51. Liu, Y, et al., Cancer Cell 33, 721-735 E728 (2018).
52. Dulak, A., et al., Nat. Genet. 45, 478-486 (2013).
53. Frankell, A, et al., Nat. Genet. 51(3):506-516 (2019).
54. Bruce W and Van Der Gaag H. Nature 199, 79-80 (1963).
55. Lessard, J, and Sauvageau, G, Nature 423, 255-260 (2003).
56. Reya T, et al., Nature 414, 105-111 (2001).
57. Kreso A, and Dick J. Cell Stem Cell 14, 275-291 (2014).
58. Thibodeau et al., Science 260, 816-819 (1993).
59. Sottoriva A, et al., Nat Genet. 47(3):209-16 (2015).
60. Ma M, et al., Dis Esophagus. 30, 1-5 (2017).
61. Ganem N, et al., Curr Opin Genet Dev. 17, 157-162 (2007).
62. Eggert U, et al., Annu Rev Biochem. 75:543-66 (2006).

### References for Methods

63. Bolger et al., Bioinformatics 30, 2114-2120 (2014).
64. Conway, T. et al., Bioinformatics 28, I172-178 (2012).
65. Li, H. & Durbin, R., Bioinformatics 26, 589-595 (2010).
66. Depristo, M. A. et al., Nat. Genet. 43, 491-498 (2011).
67. Van Der Auwera, G. A. et al., Curr. Protoc. Bioinformatics 43, 11 10 11-33 (2013).
68. Chen, X. et al., Bioinformatics 32, 1220-1222 (2016).
69. Saunders, C. T. et al., Bioinformatics 28, 1811-1817 (2012).
70. Kim, S. et al. Biorxiv (2017).
71. Mckenna, A. et al., Genome Res. 20, 1297-1303 (2010).
72. Zafar, H., et al., Genome Biol. 18, 178 (2017).
73. Chen, E. Y. et al., Bmc Bioinformatics 14, 128 (2013).

1. A method for treating a patient presenting with one or more of chronic inflammatory injury, metaplasia, dysplasia or cancer of an esophageal tissue, which method comprises administering to the patient an IAP Inhibitor agent that selectively kills or inhibits the proliferation or differentiation of pathogenic esophageal stem cells (PESC) in the esophageal tissue relative to normal regenerative stem cells of the epithelial tissue.

2. A method of reducing proliferation, survival, migration, or colony formation ability of a pathogenic esophageal stem cell (PESC) in a subject in need thereof comprising contacting the cell with a therapeutically effective amount of an IAP Inhibitor agent that selectively kills or inhibits the proliferation or differentiation of PESC relative to normal regenerative esophageal stem cells.

3. A pharmaceutical preparation for treating one or more of chronic inflammatory injury, metaplasia, dysplasia or cancer of an epithelial tissue, which preparation comprises an IAP Inhibitor agent that selectively kills or inhibits the proliferation or differentiation of pathogenic esophageal stem cells (PESC) in the esophageal tissue relative to normal regenerative esophageal stem cells.

4. (canceled)

5. A method for treating a patient presenting with one or more of esophagitis, Barrett's esophagus, esophageal dysplasia, esophageal cancer, gastric intestinal metaplasia or gastric cancer, which method comprises administering to the patient an IAP Inhibitor agent that selectively kills or inhibits the proliferation or differentiation of Barrett's Esophagus stem cells (BESC), gastric intestinal metaplasia (GIM) stem cells, esophageal cancer cells or gastric cancer cells relative to normal esophageal stem cells or stomach stem cells.

6. A method of reducing proliferation, survival, migration, or colony formation ability of a Barrett's Esophagus stem cell (BESC), gastric intestinal metaplasia (GIM) stem cells, esophageal cancer cells and gastric cancer cells in a subject in need thereof comprising contacting the cell with a therapeutically effective amount of an IAP Inhibitor agent that selectively kills or inhibits the proliferation or differentiation of BESC, GIM stem cells, esophageal cancer cells or gastric cancer cells relative to normal esophageal stem cells or stomach stem cells.

7. A pharmaceutical preparation for treating one or more of esophagitis, Barrett's esophagus, esophageal dysplasia esophageal cancer, gastric intestinal metaplasia, or gastric cancer, which preparation comprises an IAP Inhibitor agent that selectively kills or inhibits the proliferation or differentiation of Barrett's Esophagus stem cells (BESC), GIM stem cells, esophageal cancer cells or gastric cancer cells relative to normal esophageal stem cells or stomach stem cells.

8. A drug eluting device for treating one or more of esophagitis, Barrett's esophagus, esophageal dysplasia esophageal cancer, gastric intestinal metaplasia, or gastric cancer, which device comprises drug release means including an IAP Inhibitor agent that selectively kills or inhibits the proliferation or differentiation of Barrett's Esophagus stem cells (BESC), gastric intestinal metaplasia stem cells, esophageal cancer cells, or gastric cancer cells relative to normal esophageal stem cells or stomach stem cells, which device when deployed in a patient positions the drug release means proximal to the luminal surface of the esophagus or in the stomach region and releases the agent in an amount sufficient to achieve a therapeutically effective exposure of the luminal surface to the agent.

9. The method of claim 5 for the treatment of Barrett's Esophagus, Gastric Intestinal Metaplasia, esophageal adenocarcinoma, or gastric cancer.

10. (canceled)

11. The method of claim 5, wherein the IAP Inhibitor agent is administered during or after endoscopic ablation therapy, such as radiofrequency ablation, photodynamic therapy or cryoablation of esophageal tissue and gastric tissue.

12. The method of claim 5, wherein the IAP Inhibitor agent is administered by submucosal injection of esophageal tissue and gastric tissue.

13. (canceled)

14. The method of claim 5, wherein the IAP Inhibitor agent is formulated as part of a bioadhesive formulation.

15. The method of claim 5, wherein the IAP Inhibitor agent is formulated as part of a drug-eluting particle, drug eluting matrix or drug-eluting gel.

16. The method of claim 5, wherein the IAP Inhibitor agent is administered by topical application to the esophageal tissue and gastric tissue.

17-19. (canceled)

20. The method of claim 5, wherein the IAP Inhibitor agent is co-administered with an analgesic, an anti-infective or both.

21. (canceled)

22. The preparation of claim 7, wherein the IAP Inhibitor agent is formulated as a liquid for oral delivery to the epithelial tissue, such as the esophagus and stomach.

23. (canceled)

24. The device of claim 4, wherein the drug eluting device is a drug eluting stent or balloon catheter having a surface coating including the IAP Inhibitor agent.

25-26. (canceled)

27. The method of claim 5, wherein the IAP Inhibitor agent inhibits the proliferation or differentiation of BESCs, or kills BESCs, with an IC₅₀ of 10⁻⁶ M or less, more preferably 10⁻⁷ M or less, 10⁻⁸ M or less or 10⁻⁹M or less.

28. (canceled)

29. The method of claim 5, wherein the IAP Inhibitor agent is an XIAP Inhibitor.

30. The method of claim 5, wherein the IAP Inhibitor agent is SM-164 or AZD5582.

31. The method of claim 5, further comprising combining the agent with a second drug agent that selectively promotes proliferation of normal regenerative esophageal stem cells in the target with an EC₅₀ at least 5 times more potent than for BESCs in the target tissue, more preferably with an EC₅₀ 10 times, 50 times, 100 times or even 1000 times more potent than for BESCs.

32. (canceled)

33. The method of claim 5, wherein the second drug agent is a TAK1 inhibitor or a RET inhibitor.

34. The method of claim 5, wherein the second drug agent is pan-inhibitor of ABL kinase inhibitor selected from the group consisting of imatinib, nilotinib, dasatinib, bosutinib and ponatinib or pharmaceutically acceptable salt(s), solvate(s), and/or hydrate(s) thereof, and is preferably ponatinib or pharmaceutically acceptable salt(s), solvate(s), and/or hydrate(s) thereof.

35. The method of claim 5, wherein the second drug agent is FLT kinase inhibitor selected from the group consisting of quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof, and is preferably quizartinib or pharmaceutically acceptable salt(s), solvate(s), and/or hydrate(s) thereof.

36. The method of claim 5, wherein the TAP inhibitor is a bivalent SMAC mimetic and the second agent is a TAK1 inhibitor or a RET inhibitor.

37. The method of claim 5, further comprising combining the agent with a one or more antitussives, antihistamines, antipyretics, analgesics, anti-infective agents and/or chemotherapeutic agents.

38. The method of claim 31, wherein the agent and the second agent are administered to the patient as separate formulations.

39. The method of claim 21, wherein the agent and the second agent are co-formulated together.

40. The method of claim 37, wherein the agent and the one or more antitussives, antihistamines, antipyretics, analgesics, anti-infective agents and/or chemotherapeutic agents are co-formulated together.

41-44. (canceled)

45. A drug eluting device comprising drug release means including an IAP Inhibitor agent, which device when deployed in a patient positions the drug release means proximal to a target epithelial tissue to be treated and releases the IAP Inhibitor agent in an amount sufficient to achieve a therapeutically effective exposure of the target tissue to the IAP Inhibitor agent.

46. (canceled)
